(19) United States
(12) Patent Application Publication
(10) Pub. No.: US 2016/0045143 A1
(43) Pub. Date: Feb. 18, 2016

(54) APPARATUS FOR NONINVASIVELY MEASURING BIO-ANALYTE AND METHOD OF NONINVASIVELY MEASURING BIO-ANALYTE

(71) Applicant: SAMSUNG ELECTRONICS CO., LTD., Suwon-si (KR)

(72) Inventors: Joonhyung LEE, Yongin-si (KR); Sangkyu KIM, Yongin-si (KR)

(73) Assignee: SAMSUNG ELECTRONICS CO., LTD., Suwon-si (KR)

(21) Appl. No.: 14/744,720

(22) Filed: Jun. 19, 2015

(30) Foreign Application Priority Data
Aug. 12, 2014 (KR) 10-2014-0104534

(51) Int. Cl. A61B 5/1455; A61B 5/145

(52) U.S. Cl. CPC A61B 5/1455; A61B 5/14546

(57) ABSTRACT

Provided are an apparatus for noninvasively measuring a bio-analyte and a method of noninvasively measuring a bio-analyte. The apparatus may include a sensor configured to obtain information of a first material from a first body part of a subject, and a processor configured to obtain information about a second material in a second body part of the subject based on a correlation between the first material and the second material and the information of the first material.

### CROSS-REFERENCE TO RELATED APPLICATION

This application claims priority from Korean Patent Application No. 10-2014-0104534, filed on Aug. 12, 2014, in the Korean Intellectual Property Office, the disclosure of which is incorporated herein in its entirety by reference.

### BACKGROUND

1. Field

Apparatuses and methods consistent with exemplary embodiments relate to measuring a bio-analyte in a non-invasive manner.

2. Description of the Related Art

As medical science has progressed and an average life expectancy has increased, the interest in health care has increased. Accordingly, the interest in medical devices has also increased. The interest in small-medium medical devices that are used in public places and small medical devices and health care devices that are possessed or carried by individuals as well as various medical devices that are used in hospitals or health examination centers has increased.

An invasive measuring method is often used for medical devices or medical examination. An invasive measuring method may be performed by collecting blood of a subject and measuring and analyzing the collected blood. A health condition of the subject may be examined by measuring a concentration of a specific material in the blood. However, the invasive measuring method has disadvantages in that the subject feels pain when the blood is collected and reagents and colorimetric assays that react with the specific material of the blood have to be used when the blood is analyzed.

### SUMMARY

Exemplary embodiments address at least the above problems and/or disadvantages and other disadvantages not described above. Also, the exemplary embodiments are not required to overcome the disadvantages described above, and may not overcome any of the problems described above.

According to an aspect of exemplary embodiment, there is provided an apparatus for noninvasively measuring a bio-analyte including: a sensor configured to obtain information of a first material from a first body part of a subject, and a processor configured to obtain information about a second material in a second body part of the subject based on a correlation between the first material and the second material and the information of the first material.

The first body part and the second body part may exist at different depths from a surface of a skin of the subject.

The first body part may be tissue of the subject and the second body part may be blood of the subject.

The first body part may be an epidermis or a dermis of the subject.

The sensor may be an optical data obtainer including: a light source that emits light to the first body part of the subject; and a detector that detects light that is reflected or scattered by the first body part.

The optical data obtainer may further include a spectrometer configured to disperse the light that is reflected or scattered by the first body part and transmit the reflected or scattered light to the detector.

The sensor may be an optical data obtainer including an infrared (IR) spectrometer configured to obtain the information of the first material from the first body part.

The IR spectrometer may be a mid-IR (MIR) spectrometer using MIR rays.

The MIR rays may have a wavelength ranging from about 2.5 μm to about 8 μm.

The IR spectrometer may be an attenuated total reflectance (ATR)-IR spectrometer.

The IR spectrometer may be a Fourier transform (FT)-IR spectrometer.

The IR spectrometer may be an ATR-Fourier transform infrared (FTIR) spectrometer.

The sensor may include a Raman spectrometer configured to obtain raw data including the information of the first material from the first body part.

The first material may include creatine or a constituent material of creatine, and the second material may include creatinine.

The first material may include at least one from among a COOH functional group, a C═N functional group, and a C—N functional group, and the second material may include creatinine.

The sensor may be configured to obtain IR spectrum data about the first body part in order to obtain the information of the first material, and may determine an intensity value corresponding to at least one wavenumber range from among 1690 to 1760 cm⁻¹, 1650 to 1720 cm⁻¹, and 1020 to 1250 cm⁻¹ in the IR spectrum data.

According to another aspect of an exemplary embodiment, there is provided an apparatus for noninvasively measuring a bio-analyte including: a sensor that noninvasively obtains information of a first material from a skin of a subject; and a data processor configured to derive information of a second material in blood of the subject from the information about the first material.

The sensor may include any one from among an infrared (IR) spectrometer, an attenuated total reflectance (ATR)-IR spectrometer, a Fourier transform (FT)-IR spectrometer, and an ATR-Fourier transform infrared (FTIR) spectrometer.

The measurer may use a mid-IR (MIR) light source.

The measurer may include a Raman spectrometer.

According to another aspect of exemplary embodiment, a method of noninvasively measuring a bio-analyte includes: obtaining, by an sensor, information of a first material from a first body part of a subject; and deriving information of a second material that exists in a second body part of the subject based on a correlation between the first material and the second material, by a processor and the information of the first material.

The first body part and the second body part may exist at different depths from a surface of a skin of the subject.

The first body part may be tissue of the subject and the second body part may be blood of the subject.

The obtaining the information about the first material may include analyzing the first body part by using light.

The obtaining the information about the first material may include performing infrared (IR) spectroscopic analysis on the first body part.

The IR spectroscopic analysis may be performed by using mid-IR (MIR) rays.

The IR spectroscopic analysis may be performed by using one from among an IR spectrometer, an attenuated total reflectance (ATR)-IR spectrometer, a Fourier transform (FT)-IR spectrometer, and an ATR-Fourier transform infrared (FTIR) spectrometer.

The obtaining the information about the first material may include performing Raman spectroscopic analysis on the first body part.

The method may further include obtaining the correlation between the first material and the second material, wherein the deriving the information of the second material is performed by using an algorithm based on the correlation.

The first material may include creatine or a constituent material of creatine, and the second material may include creatinine.

According to another aspect of an exemplary embodiment, there is provided a method of noninvasively measuring a bio-analyte (hereinafter, referred to as a noninvasive measuring method) including: obtaining information of a first material from tissue of a subject; and deriving information of a second material in the blood of the subject from the information of the first material and a correlation between the first material in tissue of a plurality of samples and the second material in blood of the plurality of samples.

The noninvasive measuring method may further include obtaining the correlation. The obtaining the correlation may include: obtaining data that indicates an amount of the first material in the tissue of each of the plurality of samples; obtaining data that indicates an amount of the second material in the blood of each of the plurality of samples; and obtaining a relationship between the data about the first material and the data about the second material.

The obtaining of the data about the first material in the tissue of each of the plurality of samples may include: obtaining spectrum data by using spectroscopy in the tissue of each of the plurality of samples; and determining an intensity value corresponding to the first material in the spectrum data.

The obtaining the data of the first material in the tissue of each of the plurality of samples further may include performing normalization by dividing the intensity value corresponding to the first material by an intensity value corresponding to a reference wavenumber.

The obtaining of the information about the first material from the tissue of the subject may include performing infrared (IR) spectroscopic analysis on the tissue of the subject.

The IR spectroscopic analysis may be performed by using mid-IR (MIR) rays.

The IR spectroscopic analysis may be performed by using any one from among an IR spectrometer, an attenuated total reflectance (ATR)-IR spectrometer, a Fourier transform (FR)-IR spectrometer, and an ATR-Fourier transform infrared (FTIR) spectrometer.

The obtaining the information about the first material from the tissue of the subject may include performing Raman spectroscopic analysis on the tissue of the subject.

The first material may include creatine or a constituent material of creatine and the second material may include creatinine.

According to another aspect of an exemplary embodiment, there is provided an apparatus for measuring a bio-analyte of a subject including: a spectrometer configured to collect light reflected from an area of interest on a skin surface of the subject; and a processor configured to analyze the collected light to determine a concentration of a first component in the skin of the subject and determine a concentration of a second component beneath the skin surface of the subject based on a correlation between the concentration of the first component and the concentration of the second component.

The processor may be further configured to determine the concentration of the second component based on a correlation table that includes data on intensities of absorption peaks of a plurality of functional groups of the first material and wavenumbers of the absorption peaks.

The plurality of functional groups may include at least one from among a COOH functional group, a C═N functional group, and a C—N functional group.

### BRIEF DESCRIPTION OF THE DRAWINGS

The above and/or other aspects will be more apparent by describing certain exemplary embodiments, with reference to the accompanying drawings, in which:

FIG. 1 is a conceptual view for illustrating an apparatus for noninvasively measuring a bio-analyte (hereinafter, referred to as a noninvasive measuring apparatus) according to an exemplary embodiment;

FIG. 2 is a cross-sectional view for illustrating a body part of a subject that is measured by using the noninvasive measuring apparatus;

FIG. 3 is a block diagram for illustrating a configuration of a noninvasive measuring apparatus, according to an exemplary embodiment;

FIG. 4 is a block diagram for illustrating a configuration of a noninvasive measuring apparatus, according to another exemplary embodiment;

FIG. 5 is a block diagram for illustrating a configuration of a noninvasive measuring apparatus, according to another exemplary embodiment;

FIG. 6 is a view illustrating a structure of an interferometer of FIG. 5, according to an exemplary embodiment;

FIG. 7 is a block diagram for illustrating a configuration of a noninvasive measuring apparatus, according to another exemplary embodiment;

FIG. 8 is a block diagram for illustrating a configuration of a noninvasive measuring apparatus, according to another exemplary embodiment;

FIG. 9 is a view for illustrating a light source that may be used in a noninvasive measuring apparatus, according to an exemplary embodiment;

FIG. 10 is a block diagram for illustrating a configuration of a noninvasive measuring apparatus, according to another exemplary embodiment;

FIG. 11 is a block diagram for illustrating a configuration of a processor that may be used in a noninvasive measuring apparatus, according to an exemplary embodiment;

FIG. 12 is a block diagram for illustrating a processor and an output unit that may be used in a noninvasive measuring apparatus, according to an exemplary embodiment;

FIG. 13 is a graph illustrating a correlation between a value of a first material that is detected by a noninvasive measuring apparatus and a value of a second material that is output by the noninvasive measuring apparatus, according to an exemplary embodiment;

FIG. 14 is a graph illustrating a correlation between a value of a first material that is detected by a noninvasive measuring apparatus and a value of a second material that is output by the noninvasive measuring apparatus, according to another exemplary embodiment;

FIG. 15 illustrates a chemical structure of creatine;

FIG. 16 illustrates a chemical structure of creatinine;

FIGS. 17 through 27 are infrared (IR) spectrum data that is obtained from tissue of the skin of a plurality of samples;

FIG. 28 is a graph for illustrating a method of extracting information of creatine in the IR spectrum data of FIG. 17;

FIG. 29 is a graph illustrating a correlation between different materials of a subject, according to an exemplary embodiment;

FIG. 30 is a graph illustrating a correlation between different materials of a subject, according to another exemplary embodiment;

FIG. 31 is a flowchart for illustrating a method of noninvasively measuring a bio-analyte (hereinafter, referred to as a noninvasive measuring method), according to an exemplary embodiment;

FIG. 32 is a flowchart for illustrating a noninvasive measuring method according to another exemplary embodiment;

FIG. 33 is a flowchart for illustrating a noninvasive measuring method according to another exemplary embodiment;

FIG. 34 is a block diagram illustrating a noninvasive measuring apparatus according to an exemplary embodiment;

FIG. 35 is a block diagram illustrating a noninvasive measuring apparatus according to another exemplary embodiment;

FIG. 36 is a block diagram illustrating a noninvasive measuring apparatus according to another exemplary embodiment;

FIG. 37 is a block diagram illustrating a noninvasive measuring apparatus according to another exemplary embodiment;

FIG. 38 is a block diagram illustrating a noninvasive measuring apparatus according to another exemplary embodiment;

FIG. 39 is a block diagram illustrating a noninvasive measuring apparatus according to another exemplary embodiment; and

FIG. 40 is a conceptual view illustrating an example in which relative positions of a subject and the noninvasive measuring apparatus of FIG. 1 are changed.

### DETAILED DESCRIPTION OF EXEMPLARY EMBODIMENTS

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. As used herein the term “and/or” includes any and all combinations of one or more of the associated listed items.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of exemplary embodiments.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” may encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of exemplary embodiments. As used herein, the singular forms “a,” “an”, and “the” are intended to include the plural forms as well, unless the context clearly indicates otherwise. It will be further understood that the terms “comprises” and/or “comprising,” when used in this specification, specify the presence of stated features, integers, steps, operations, elements, components, and/or groups but do not preclude the presence or addition of one or more other features, integers, steps, operations, elements, components, and/or groups thereof.

Exemplary embodiments are described herein with reference to cross-sectional illustrations that are schematic illustrations of idealized embodiments (and intermediate structures) of exemplary embodiments. As such, variations from the shapes of the illustrations as a result, for example, of manufacturing techniques and/or tolerances, are to be expected. Thus, exemplary embodiments should not be construed as limited to the particular shapes of regions illustrated herein but are to include deviations in shapes that result, for example, from manufacturing. For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of exemplary embodiments.

Unless otherwise defined, all terms (including technical and scientific terms) used herein have the same meaning as commonly understood by one of ordinary skill in the art to which exemplary embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

FIG. 1 is a conceptual view for illustrating an apparatus for noninvasively measuring a bio-analyte (hereinafter, referred to as a noninvasive measuring apparatus) 100 according to an exemplary embodiment. The term ‘bio-analyte’ may refer to a constituent material of a body of a living creature, such as a human or an animal or a component of the constituent material. The bio-analyte may also refer to a constituent material of a subject S1 to be measured by the noninvasive measuring apparatus 100. For example, the bio-analyte may be a material that is included in tissue or blood of the subject S1 or a component of the material. A first material A and a second material B which will be explained below may be included in the bio-analyte. Also, the subject S1 itself may be considered to be the bio-analyte. The term ‘bio-analyte’ may encompass a general ‘target analyte’ that is used in medical fields or diagnosis/measurement fields.

FIG. 1 shows the noninvasive measuring apparatus 100 that measures the subject S1 in a non-invasive manner. The noninvasive measuring apparatus 100 may be an apparatus that obtains information about a first material A from a first body part P1 of the subject S1 and outputs information about a second material B that exists in a second body part P2 of the subject S1 based on the information about the first material A. In other words, the noninvasive measuring apparatus 100 may be configured to obtain the information about the first material A in the first body part P1 by detecting and measuring the first material A and to output the information about the second material B of the second body part P2 based on the information about the first material A.

The first body part P1 and the second body part P2 may differ, and the first material A and the second material B may differ. The first body part P1 and the second body part P2 may exist at different depths from a surface (detected surface) SS1 of the subject S1. For example, the first body part P1 may exist at a first depth d1 from the surface SS1 of the subject S1, and the second body part P2 may exist at a second depth d2, which is greater than the first depth d1, from the surface SS1. Accordingly, the second body part P2 may be farther than the first body part P1 from the noninvasive measuring apparatus 100. The surface SS1 may be the surface of the skin of the subject S1. For example, the first body part P1 may be tissue of the skin and the second body part P2 may be blood in a blood vessel BV1. The first body part P1 may be tissue of a body part (for example, an organ) other than the skin and the second body part P2 may not be blood.

There may be a correlation between the information about the first material A in the first body part P1 and the information about the second material B in the second body part P2. The noninvasive measuring apparatus 100 may be configured to calculate the information about the second material B from the information about the first material A according to an algorithm based on the correlation. The correlation and the information calculation (data processing) using the correlation will be explained below in detail.

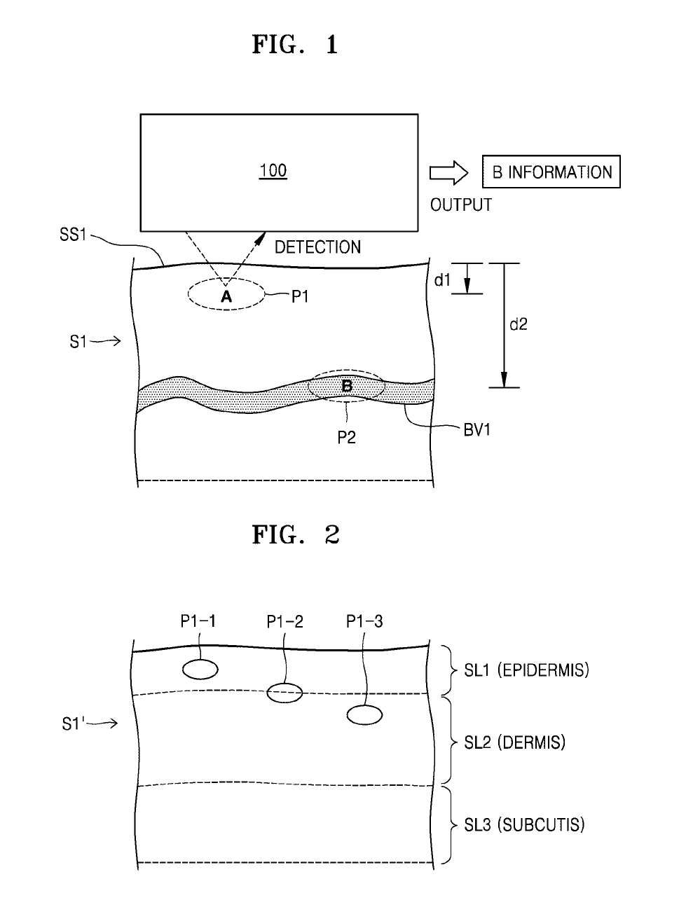

FIG. 2 is a cross-sectional view for illustrating a measured body part of a subject S1′ that is measured by using the noninvasive measuring apparatus 100.

As shown in FIG. 2, the skin of the subject S1′ may include an epidermis SL1 and a dermis SL2. The epidermis SL1 exists on an outer portion of the skin and the dermis SL2 exists under the epidermis SL1. A subcutis SL3 may exist under the dermis SL2. A blood vessel (not shown) may exist in the subcutis SL3, and a blood vessel (not shown) or a capillary may also exist in the dermis SL2. When the subject S1′ is measured by using the noninvasive measuring apparatus 100 of FIG. 1 according to an exemplary embodiment, the subject S1′ may be measured through the epidermis SL1 or the dermis SL2. First through third measured body parts P1-1, P1-2, and P1-3 of FIG. 2 are regions measured at different depths. The first through third measured body parts P1-1, P1-2, and P1-3 may correspond to the first body part P1 of FIG. 1. In other words, the first through third measured body parts P-1, P1-2, and P1-3 may be regions that are directly measured/detected by the noninvasive measuring apparatus 100 of FIG. 1. A region of the epidermis SL1 such as the first measured body part P1-1 may be measured, a region, including a part of the epidermis SL1 and a part of the dermis SL2 such as the second measured body part P1-2, may be measured, or a region of the dermis SL2 such as the third measured body part P1-3 may be measured.

A depth/range of a measured body part (e.g., the first body part P1 of FIG. 1) may vary according to a measurement method and/or a measurement unit that is used by the noninvasive measuring apparatus 100 of FIG. 1. When the noninvasive measuring apparatus 100 (see FIG. 1) performs measurements by using an infrared (IR) spectrometer, a depth and/or a range of the measured body part (e.g., the first body part P1 of FIG. 1) may vary according to a wavelength of IR rays that are used in the IR spectrometer. When the IR spectrometer uses mid-IR (MIR) rays, in other words, when the IR spectrometer is an MIR spectrometer, the measured body part (e.g., the first body part P1 of FIG. 1) may be a region of the epidermis SL1 or may include a region of the epidermis SL1 and a part of the dermis SL2. The MIR rays may have a wavelength ranging from about 2.5 μm to about 8 μm, and may have a skin penetration depth ranging from about 50 μm to about 100 μm. The MIR rays may be used to analyze a molecular structure of solid matter, liquid matter, or gaseous matter and may be effectively used to identify and quantize a component of a complex material by forming a narrow and sharp peak in spectrum data.

The noninvasive measuring apparatus 100 of FIG. 1 may perform measurements by using near-IR (NIR) rays. NIR rays have a wavelength ranging from about 0.75 μm to about 1.4 μm. Thus, when the noninvasive measuring apparatus 100 performs measurements by using a NIR spectrometer, a scope of a measured body part (e.g., the first body part P1 of FIG. 1) may be larger than that when the MIR rays are used. When the NIR rays are used, not only a region of the epidermis SL1 but also a region of the dermis SL2 may be measured. Exemplary embodiments are not limited to using MIR rays and NIR rays. For example, the noninvasive measuring apparatus 100 may use short-wavelength IR (SIR) rays within a bandwidth between about 1.4 μm and about 2.5 μm or long-wavelength IR (LIR) rays within a bandwidth between about 8 μm and about 15 μm.

The noninvasive measuring apparatus 100 of FIG. 1 may perform measurements by using a Raman spectrometer. The Raman spectrometer uses a laser source, and a depth and/or a range of a measured body part (e.g., the first body part P1 of FIG. 1) may vary according to a wavelength of a laser that is generated by the laser source. When the Raman spectrometer is used, not only a region of the epidermis SL1 but also a region of the dermis SL2 may be measured.

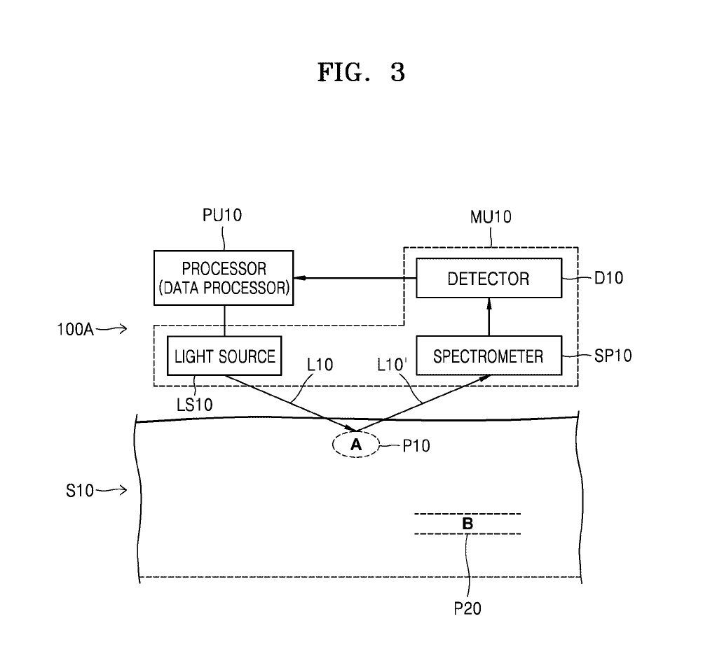

FIG. 3 is a block diagram for illustrating a configuration of a noninvasive measuring apparatus 100A, according to an exemplary embodiment.

As shown in FIG. 3, the noninvasive measuring apparatus 100A may include a measurer MU10 that obtains raw data including information about the first material A from a first body part P10 of a subject S10. The measurer MU10 may be referred to as a ‘data obtainer’. Also, the noninvasive measuring apparatus 100A may include a processor PU10. The processor PU10 may include a ‘data processor’ that calculates/derives information about the second material B in a second body part P20 of the subject S10 based on the information about the first material A. The processor PU10 may calculate and/or derive the information about the second material B from the information about the first material A by using the processor PU10. In this case, an algorithm based on a correlation between the first material A and the second material B may be used. The first body part P10 of FIG. 3 may correspond to the first body part P1 of FIG. 1 and the first through third measured body parts P1-1, P1-2, and P1-3 of FIG. 2, and the second body part P20 of FIG. 3 may correspond to the second body part P2 of FIG. 1.

The measurer MU10 may measure the first body part P10 by using light. In this case, the measurer MU10 may include a light source LS10 that emits light L10 to the first body part P10 and a detector D10 that detects light L10′ that is emitted from the light source LS10 and is reflected or scattered by the first body part 10. The measurer MU10 may further include a spectrometer SP10 that disperses the light L10′ that is reflected or scattered by the first body part P10. The light that is dispersed by the spectrometer SP10 may be detected by the detector D10. Raw data about the first body part P10 may be obtained by using the measurer MU10. The raw data may include the information about the first material A.

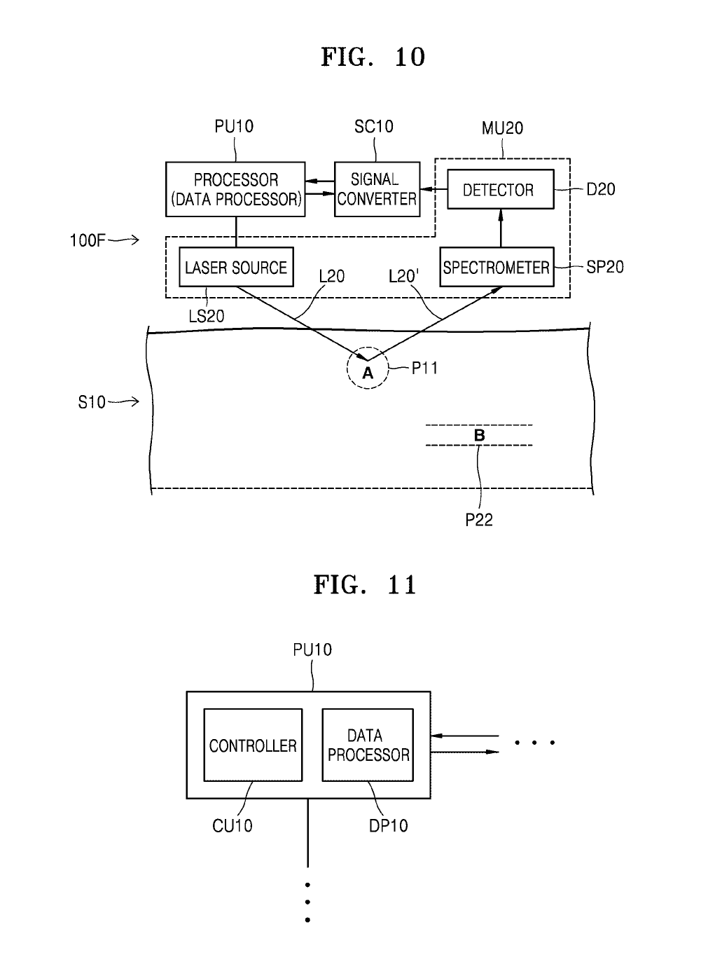

The measurer MU10 may have, for example, a structure of an IR spectrometer. In this case, the light source LS10 may be an IR source, and the light L10 that is emitted by the light source LS10 to the first body part P10 may be IR rays. The IR spectrometer may be an IR sensor. The IR spectrometer may be an MIR spectrometer using MIR rays. In this case, the light source LS10 may be an MIR source. The light L10 may be MIR rays. The MIR rays may have a wavelength ranging from about 2.5 μm to about 8 μm and may have a skin penetration depth ranging from about 50 μm to about 100 μm. The MIR rays may be used to analyze a molecular structure of solid matter, liquid matter, or gaseous matter, and may be used to identify and quantize a component of a complex material by forming a narrow and sharp peak in spectrum data. However, the IR spectrometer is not limited to the MIR spectrometer. The IR spectrometer may be an NIR spectrometer using NIR rays. Also, the measurer MU10 may have a structure of a measurement unit, for example, a Raman spectrometer, other than the IR spectrometer. The Raman spectrometer will be explained below with reference to FIG. 10.

The raw data that is obtained by the measurer MU10 may be transmitted to the processor PU10. The processor PU10 may extract the information about the first material A from the raw data, and may calculate and/or derive the information about the second material B in the second body part P20 based on the extracted information about the first material A. The extraction and the calculation of the information (data) may be performed by the ‘data processor’. Also, the processor PU10 may function to control an overall operation of the noninvasive measuring apparatus 100A including the measurer MU10 as well as an operation to extract and/or calculate the data. To this end, the processor PU10 may further include a ‘controller’ and may be connected to the light source LS10 and the detector D10.

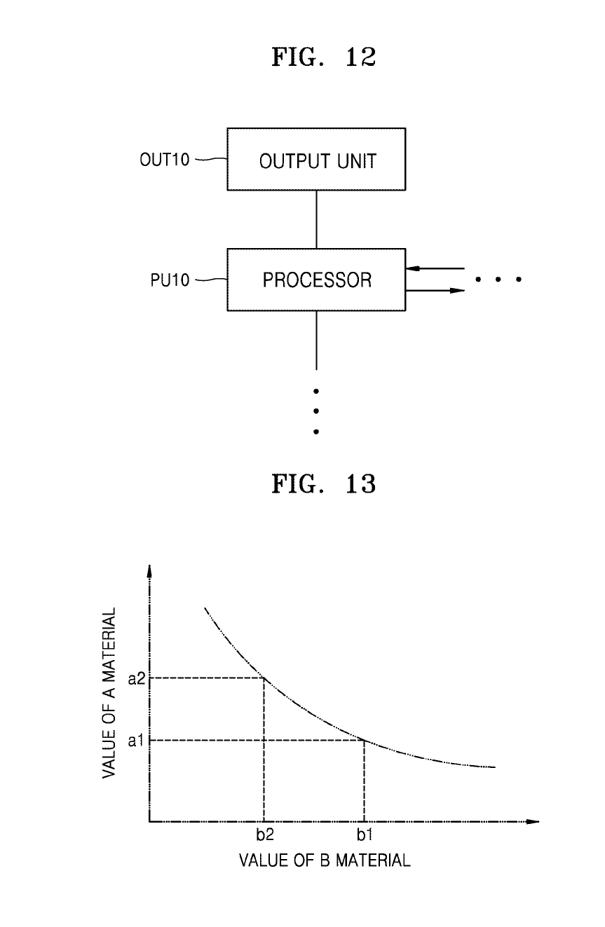

Although not shown in FIG. 3, the noninvasive measuring apparatus 100A may further include an ‘output unit’ that is connected to the processor PU10. The output unit may include, for example, a display device. The information about the second material B that is derived by the processor PU10 may be output through the output unit. The output unit will be explained below in detail with reference to FIGS. 12 and 34.

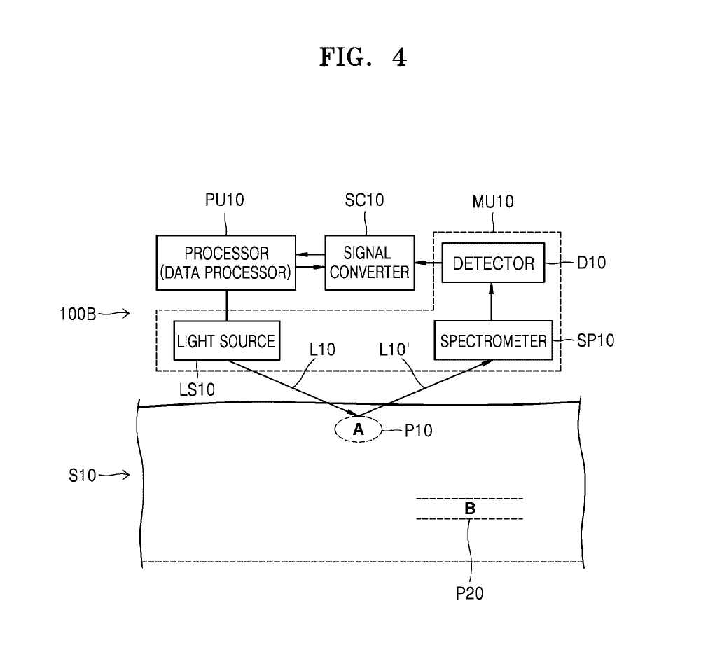

According to another exemplary embodiment, a ‘signal converter’ may be further provided between the measurer MU10 (data obtainer) and the processor PU10 (data processor) of FIG. 3. That is, as shown in FIG. 4, a noninvasive measuring apparatus 100B may further include a signal converter SC10 that is disposed between the measurer MU10 (signal obtainer) and the processor PU10 (data processor). The signal converter SC10 may include, for example, an analog front-end (AFE) circuit. The signal converter SC10 may convert an analog signal that is input from the measurer MU10 (data obtainer) into a digital signal and may transmit the digital signal to the processor PU10 (data processor). The processor PU10 may transmit a control signal to the signal converter SC10. The signal converter SC10 may operate according to the control signal of the processor PU10. Accordingly, signal transmission (that is, communication) may occur between the processor PU10 and the signal converter SC10.

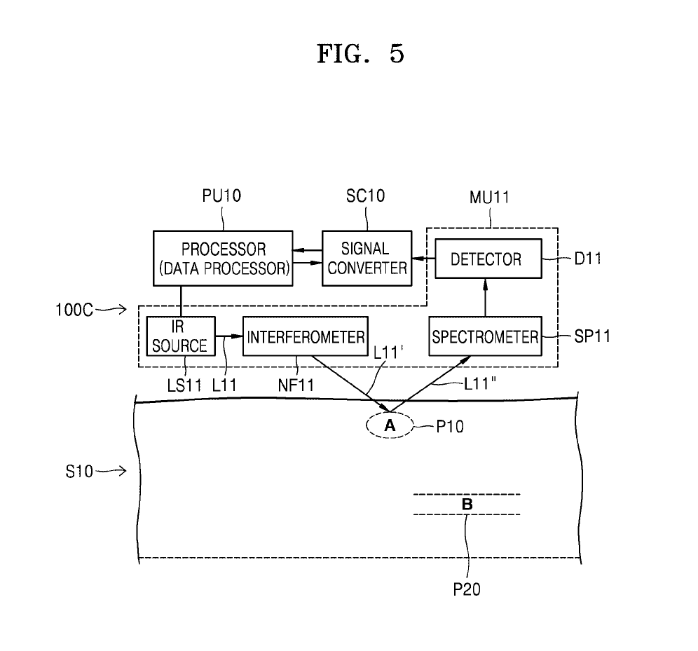

According to another exemplary embodiment, a structure of a Fourier transform (FT)-IR spectrometer as shown in FIG. 5 may be used for the measurer MU10 of FIG. 3 or 4.

FIG. 5 shows a measurer MU11 of a noninvasive measuring apparatus 100C having a structure of an FT-IR spectrometer. The measurer MU11 may include an IR source LS11 and an interferometer NF11 that is adjacent to the IR source LS11. Light L11 that is generated by the IR source LS11 may be emitted through the interferometer NF11 to the first body part P10 of the subject S10. Light that is emitted through the interferometer NF11 to the first body part P10 is denoted by L11′. Light L11″ that is reflected by the first body part P10 may be dispersed by a spectrometer SP11 and then may be detected by a detector D11. The light L11″ that is reflected by the first body part P10 may be converted by using a Fourier transform, and then may be output as a spectrum. The IR source LS11, the interferometer NF11, the spectrometer SP11, and the detector D11 may constitute the structure of the FT-IR spectrometer. If the IR source LS11 is an MIR source, the measurer MU11 may have a structure of an FT-MIR spectrometer

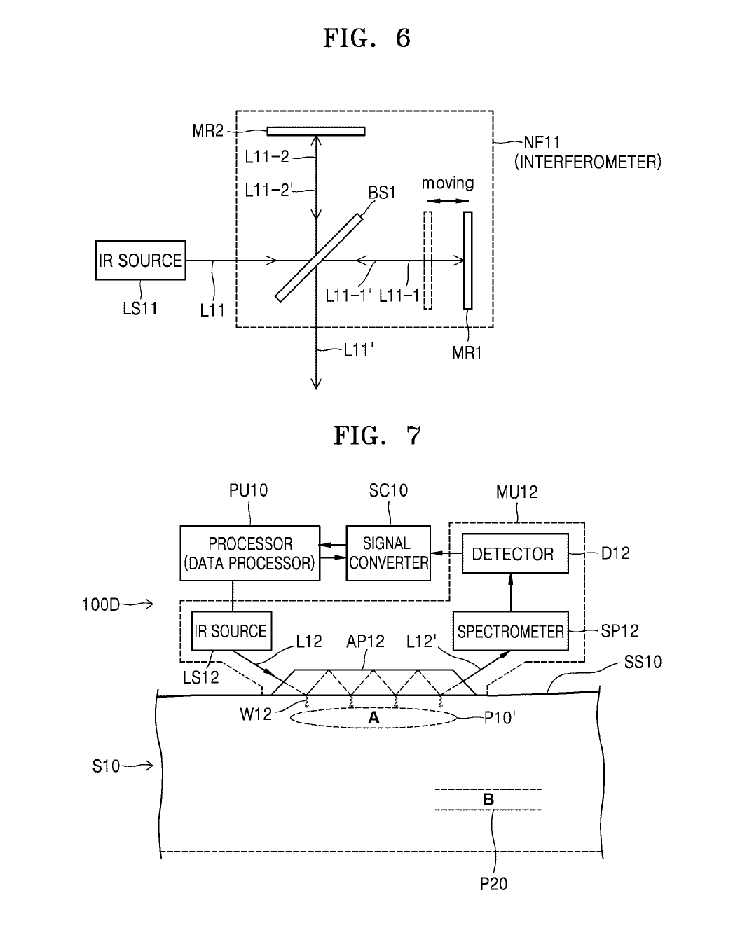

FIG. 6 is a view illustrating a structure of the interferometer NF11 of FIG. 5, according to an embodiment. As shown in FIG. 6, the interferometer NF11 may include a beam splitter BS1, a first mirror MR1, and a second mirror MR2. The light L11 that is generated by the IR source LS11 may be split by the beam splitter BS1 and may be incident on the first mirror MR1 and the second mirror MR2. Light L11-1 that passes through the beam splitter BS1 may be incident on the first mirror MR1 and light L11-2 that is reflected by the beam splitter BS1 may be incident on the second mirror MR2. The first mirror MR1 may move in a direction that is parallel to a direction in which the light L11-1 travels. The second mirror MR2 may be a fixed mirror. Light L11-1′ that is reflected by the first mirror MR1 and light L11-2′ that is reflected by the second mirror MR2 may be combined with each other through the beam splitter BS1 to form combined light L11′. More specifically, light L11-1′ is reflected on the beam splitter BS1 and light L11-2′ is partially transmitted through the beam splitter BS1, and in turn, the reflected light L11-1′ and the transmitted light L11-2′ are converged into the combined light L11′. The combined light L11′ may be emitted to the subject S10 of FIG. 5.

The measurer MU11 of FIG. 5 may have a high signal-to-noise (SNR) ratio and a high resolution because the measurer MU11 includes the interferometer NF11 and uses the Fourier transform. A configuration of the measurer MU11 and a configuration of the interferometer NF11 of FIGS. 5 and 6 are exemplary, and may be modified in various ways.

According to another exemplary embodiment, a structure of an attenuated total reflectance (ATR)-IR spectrometer may be used for the measurer MU10 of FIG. 3 or 4, as shown in FIG. 7.

FIG. 7 illustrates a measurer MU12 of a noninvasive measuring apparatus 100D including an IR source LS12 and an ATR prism AP12. The ATR prism AP12 may contact a skin surface SS10 of the subject S10. Light L12 that is generated by the IR source LS12 may be radiated onto the ATR prism AP12 and transmitted to a spectrometer SP12 through multiple internal reflections occurring in the ATR prism AP12. The light that exits the ATR prism AP12 and then is transmitted to the spectrometer SP12 is referred to as light LS12′. The transmitted light LS12′ may be dispersed by the spectrometer SP12 and then detected by a detector D12. When the light L12 is reflected in the ATR prism AP12, an evanescent wave W12 may be generated toward the subject S10. A first body part P10′ of the subject S10 that is adjacent to the evanescent wave W12 may be more effectively measured and/or detected due to the evanescent wave W12. The IR source LS12, the ATR prism AP12, the spectrometer SP12, and the detector D12 may constitute the structure of the ATR-IR spectrometer. If the IR source LS12 is an MIR source, the measurer MU12 may have a structure of a MIR-ATR spectrometer.

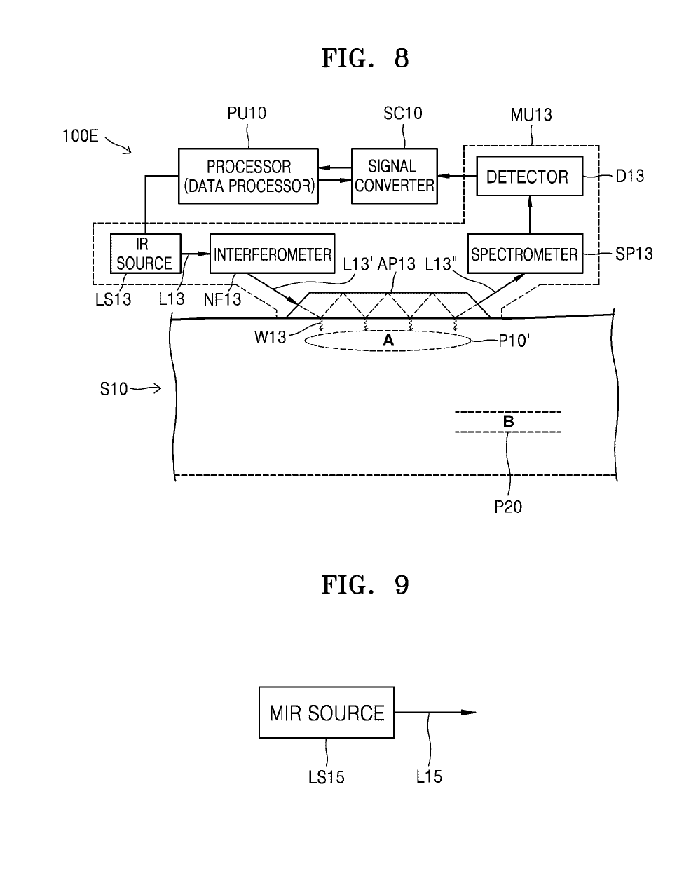

According to another exemplary embodiment, a structure of an ATR-Fourier transform infrared (FTIR) spectrometer may be used for the measurer MU10 of FIG. 3 or 4, as shown in FIG. 8.

FIG. 8 illustrates a measurer MU13 of a noninvasive measuring apparatus 100E including an IR source LS13, an interferometer NF13, an ATR prism AP13, a spectrometer SP13, and a detector D13. The interferometer NF13 may have an identical or similar structure to that of the interferometer NF11 of FIGS. 5 and 6. The ATR prism AP13 may have an identical or similar structure to that of the ATR prism AP12 of FIG. 7. The measurer MU13 may have a structure of an ATR-FTIR spectrometer. L13 denotes light that is generated by the IR source LS13, L13′ denotes light that passes through the interferometer NF13 and is emitted, and L13″ denotes light that passes through the ATR prism AP13 and is emitted. W13 denotes an evanescent wave that is generated by the ATR prism AP13. The ATR-FTIR spectrometer may have both advantages of an FT-IR spectrometer and advantages of an ATR-IR spectrometer. If when the IR source LS13 is an MIR source, the measurer MU13 may have a structure of an FT-MIR-ATR spectrometer.

The noninvasive measuring apparatuses 100A through 100E of FIGS. 3 through 8 may use an MIR source LS15 as the light sources LS10 through LS13, as shown in FIG. 9. In this case, MIR rays L15 may be emitted from the MIR source LS15, and the subject S10 may be detected and/or measured by using the MIR rays L15. The MIR rays L15 may have a wavelength ranging from about 2.5 μm to about 8 μm and may have a skin penetration depth ranging from about 50 μm to about 100 μm. The MIR rays L15 may be effectively used to identify and quantize a component of a complex material by forming a narrow and sharp peak in spectrum data. However, if necessary, a light source, such as a NIR source, other than the MIR source LS15, may be used.

In addition, configurations of the measurers MU10 through MU13 of the noninvasive measuring apparatuses 100A through 100E of FIGS. 3 through 8 may be modified in various ways. For example, positions of the spectrometers SP10 through SP13 may be changed, and the spectrometers SP10 through SP13 may not be used. Also, the spectrometers SP10 through SP13 and the detectors D10 through D13 may be integrated into single units.

According to another exemplary embodiment, a structure of a Raman spectrometer may be used for the measurer MU10 of FIG. 3 or 4, as shown in FIG. 10.

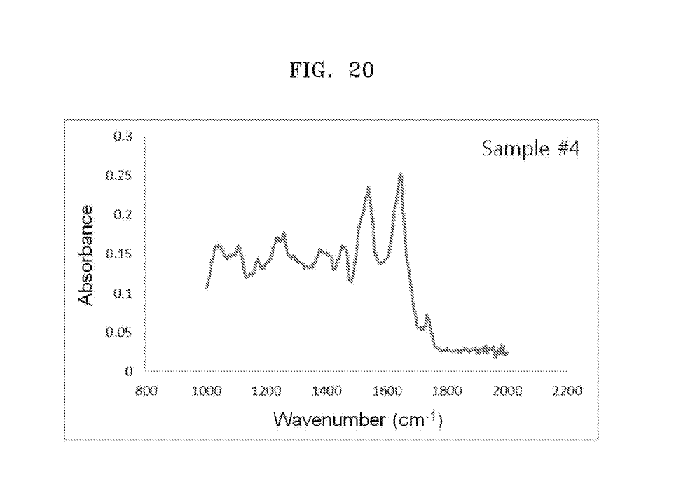

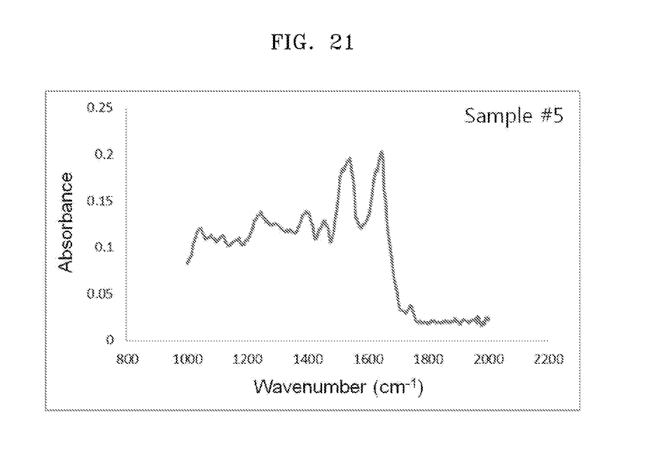

FIG. 10 shows a measurer MU20 of a noninvasive measuring apparatus 100F including a laser source LS20 as a light source. Light L20 may be emitted by the laser source LS20 to a first body part P11 of the subject S10. The light L20 may be a laser. Light L20′ that is scattered by the first body part P11 may be dispersed by a spectrometer SP20 and may be detected by a detector D20. Although FIG. 10 illustrates the detector D20 separately from the spectrometer SP20, the detector D20 may be implemented in the spectrometer SP20. The measurer MU20, including the laser source LS20, the spectrometer SP20, and the detector D20, may have a structure of a Raman spectrometer. The Raman spectrometer may analyze the first body part P11 by detecting the scattered light L20′. In this regard, the Raman spectrometer is different from an IR spectrometer that detects reflected light. Also, when the Raman spectrometer is used, a depth and a range of a measured body part (that is, the first body part P11) of the subject S10 may be different from those when an MIR spectrometer is used. A configuration of the measurer MU20, that is, the Raman spectrometer, of FIG. 20, is exemplary, and may be modified in various ways.

Information about the first material A in the first body part P11 may be obtained by measuring the first body part P11 by using the measurer MU20, and information about the second material B in a second body part P22 may be derived and output based on the information about the first material A by using the processor PU10. The signal converter SC10 may be further provided between the processor PU10 and the detector D20. Functions of the processor PU10 and the signal converter SC10 may be similar to those described with reference to FIGS. 3 and 4.

The processor PU10 that is used in each of the noninvasive measuring apparatuses 100A through 100F of FIGS. 3, 4, 5, 7, 8, and 10 may have, for example, a configuration as shown in FIG. 11.

Referring to FIG. 11, the processor PU10 may include a data processor DP10 and a controller CU10. The data processor DP10 may extract information about the first material A from raw data that is obtained by, for example, the measurer MU10 of FIG. 3. Further, the data processor DP10 may calculate and/or derive information about the second material B based on the extracted information about the first material A. The data processor DP10 may perform data processing by using an algorithm based on a correlation between the first material A and the second material B. The controller CU10 may function to control an overall operation of any of the noninvasive measuring apparatuses 100A through 100F including the measurer MU10. The processor PU10 may include a configuration/function of a central processing unit (CPU). Alternatively, the processor PU10 may have a configuration of a microcontroller unit (MCU).

The noninvasive measuring apparatuses 100A through 100F of FIGS. 3, 4, 5, 7, 8, and 10 may further include an output unit OUT10 that is connected to the processor PU10, as shown in FIG. 12.

Referring to FIG. 12, an output unit OUT10 that is connected to the processor PU10 may be further provided. The output unit OUT10 may be, for example, a display or a speaker. The output unit OUT10 may be directly connected to the processor PU10. In addition or alternatively, the output unit OUT10 and the processor PU10 may be connected to each other through wireless communication. A connection relationship between the output unit OUT10 and the processor PU10 and a configuration of the output unit OUT10 may be modified in various ways.

FIG. 13 is a graph illustrating a correlation between a value of the first material A that is detected by a noninvasive measuring apparatus and a value of the second material B that is output by the noninvasive measuring apparatus, according to an exemplary embodiment. The first material is referred to as an ‘A material’ and the second material is referred to as a ‘B material’ in FIG. 13.

As shown in FIG. 13, there may be a predetermined functional relation between a value of the A material and a value of the B material. The value of the A material and the value of the B material in the present embodiment may be substantially inversely proportional to each other. Raw data including information about the A material may be obtained by using a measurer of the noninvasive measuring apparatus, the value of the A material may be extracted from the raw data by using a data processor of the noninvasive measuring apparatus, and the value of the B material may be derived from the value of the A material. In this case, the data processor may use the functional relation (correlation). When the value of the A material is a1, the value of the B material corresponding to the value a1 may be derived to be b1 by using the functional relation (correlation). When the value of the A material is a2, the value of the B material corresponding to the value a2 may be derived to be b2 by using the functional relation (correlation). The value a2 may be greater than the value a1 and the value b2 may be less than the value b1.

FIG. 14 is a graph illustrating a correlation between a value of the first material A that is detected by a noninvasive measuring apparatus and a value of the second material B that is output by the noninvasive measuring apparatus, according to another exemplary embodiment. The first material is referred to as an ‘A material’ and the second material is referred to as a ‘B material’ in FIG. 14.

As shown in FIG. 14, there may be a predetermined functional relation between the value of the A material and the value of the B material. The value of the A material and the value of the B material in the present exemplary embodiment may be substantially proportional to each other. When the value of the A material is a1′, the value of the B material corresponding to the value a1′ may be derived to be b1′. When the value of the A material is a2′, the value of the B material corresponding to the value a2′ may be derived to be b2′. The value a2′ may be greater than the value a1′ and the value b2′ may be greater than the value b1′. The graphs of FIGS. 13 and 14 are exemplary and may be modified in various ways.

A method of obtaining a correlation between specific materials as shown in FIGS. 13 and 14 will be explained. In other words, a method of obtaining a correlation between the first material A and the second material B will now be explained in detail. The following will be explained on the assumption that the first material A is creatine in tissue and the second material B is creatinine in blood. However, the exemplary embodiment is not limited thereto and the second material B may correspond to any component included in blood, for example, glucose, lipoprotein, cholesterol, etc.

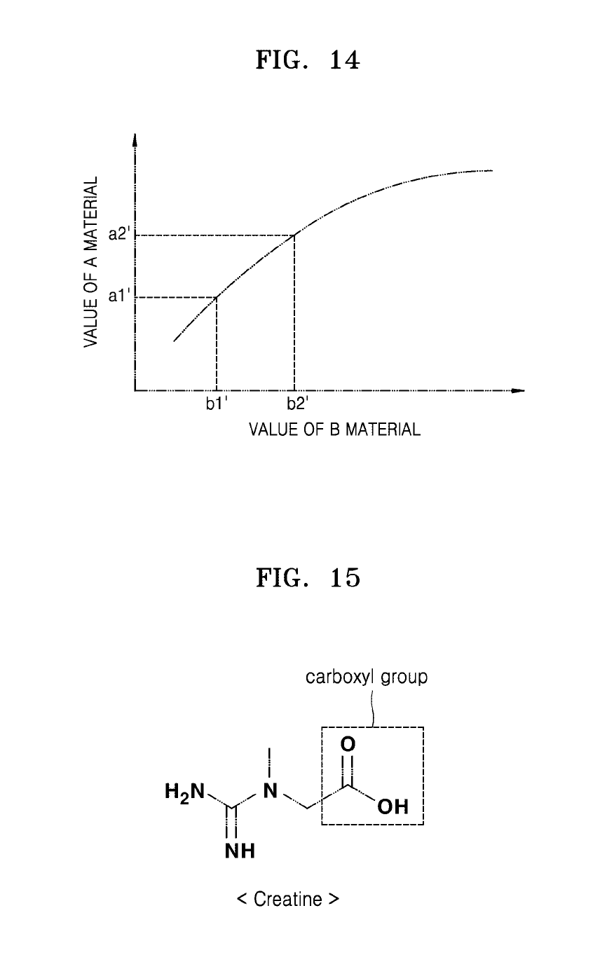

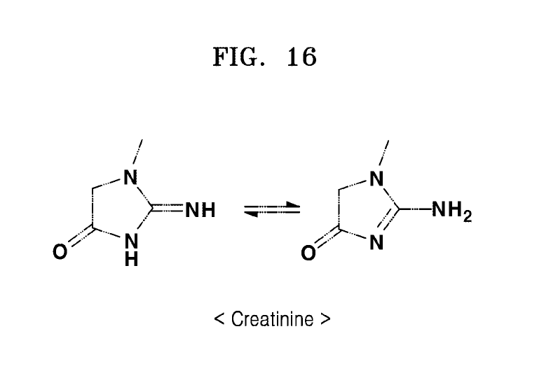

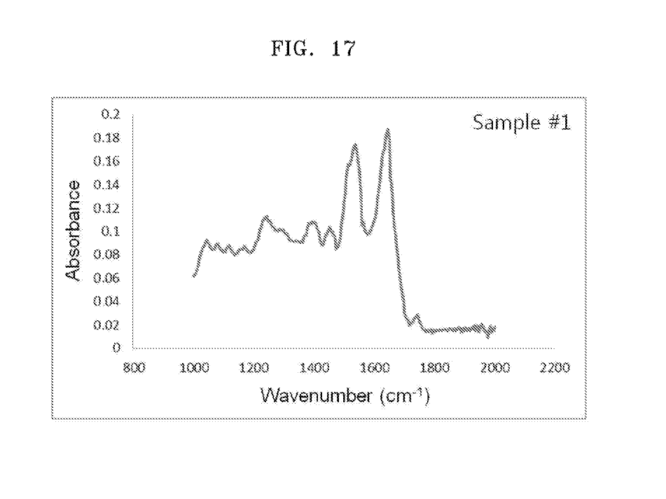

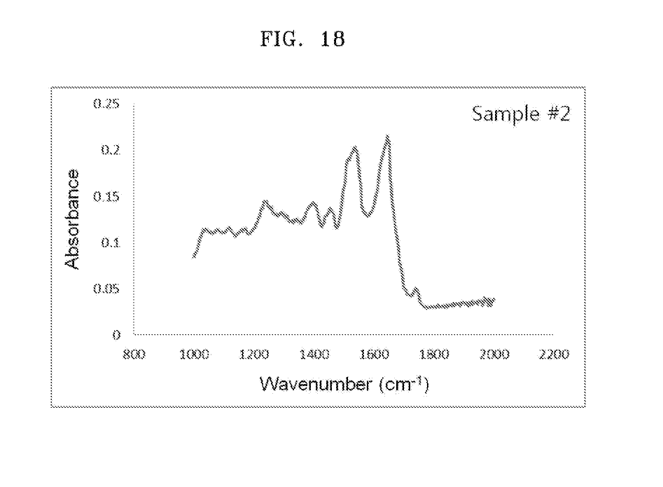

FIGS. 15 and 16 respectively illustrate chemical structures of creatine and creatinine. In the chemical structures of FIGS. 15 and 16, C indicating carbon is not shown.

As shown in FIG. 15, the creatine may include a COOH functional group, a C═N functional group, and a C—N functional group. The COOH functional group is a carboxyl group, the C═N functional group is a functional group with a carbon-nitrogen double bond, and the C—N functional group is a functional group with a carbon-nitrogen single bond.

As shown in FIG. 16, a chemical structure of the creatinine may be changed between two structures. When a position of hydrogen (H) is changed in the left chemical structure, the right chemical structure may be obtained. The creatinine may have any of the two chemical structures. The creatinine has a chemical structure that is different from that of the creatine of FIG. 15.

The creatinine, a material that is removed in blood by the kidney may be used as an indicator of kidney health. A reference value of the creatinine ranges from about 0.7 mg/dL to about 1.2 mg/dL. When a value of the creatinine in blood is high, it means that the kidney deteriorates. The creatinine may be made from the creatine. The creatinine is generated in the liver and is stored in each organ and tissue through blood. Accordingly, the amount/concentration of the creatine in tissue and the amount/concentration of the creatinine in blood have a correlation therebetween.

In order to obtain the correlation between the creatine in the tissue and the creatinine in the blood, a plurality of samples collected from a plurality of human subjects may be used. Data of the creatine in the tissue may be obtained from the plurality of samples (people) and data of the creatinine in the blood may be obtained, and then a correlation (relationship) between the two pieces of data may be obtained.

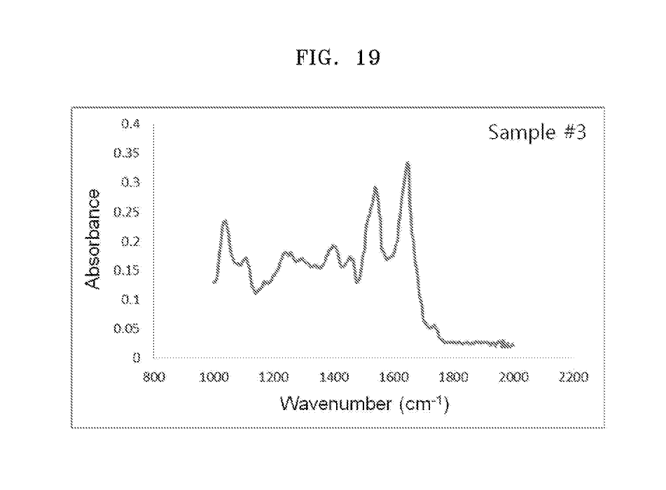

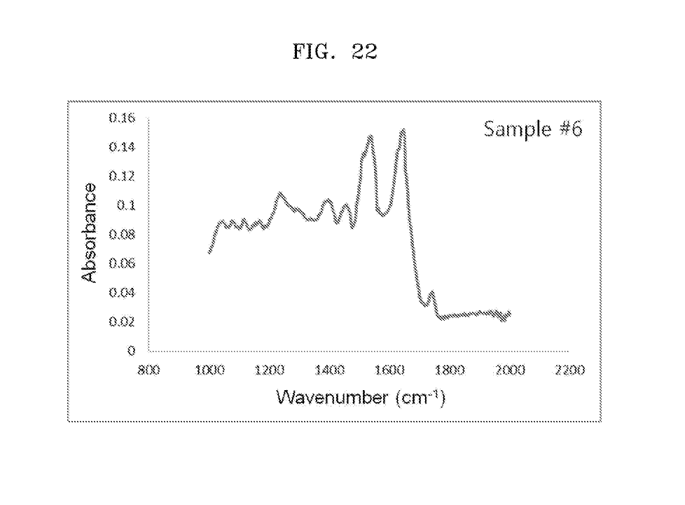

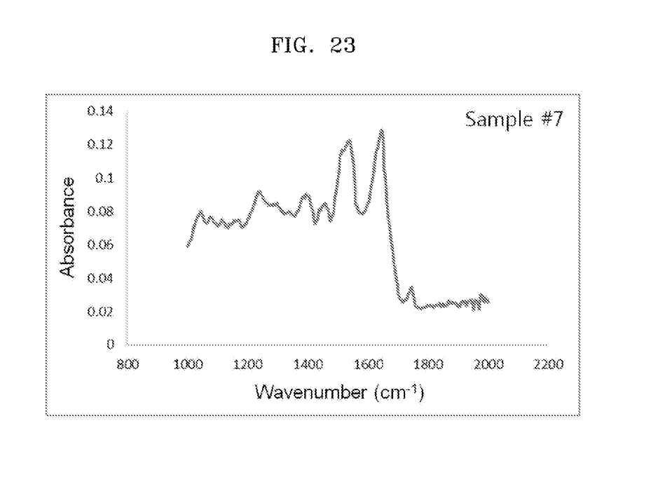

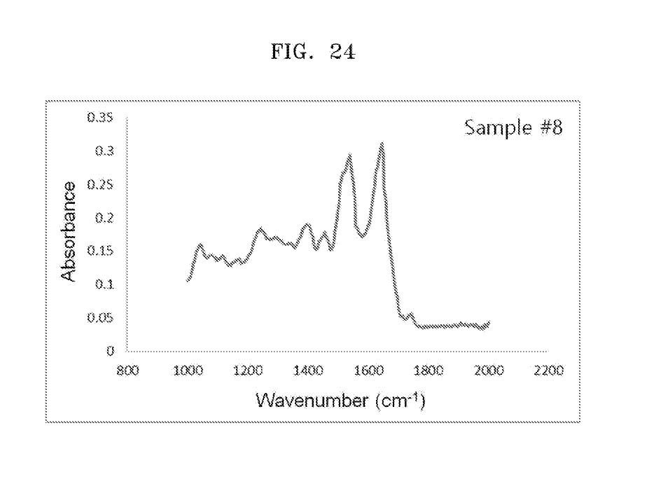

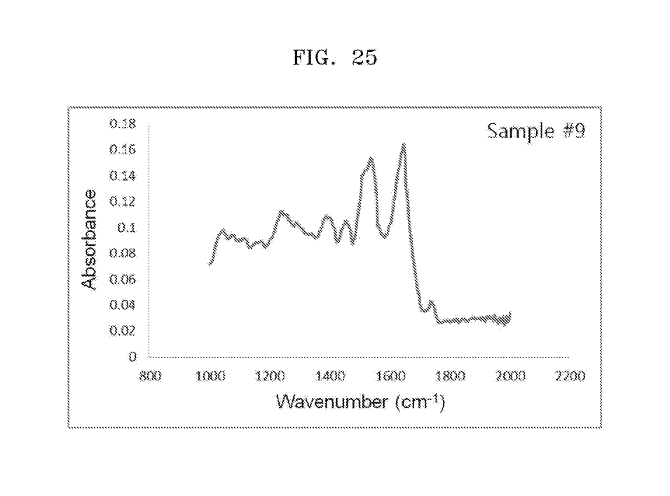

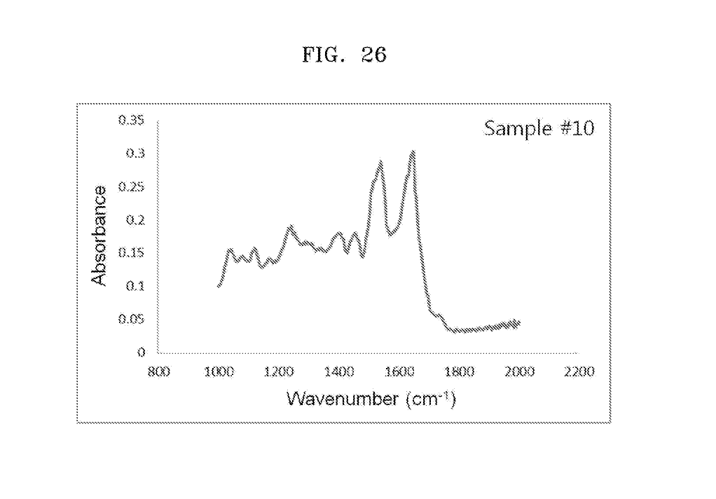

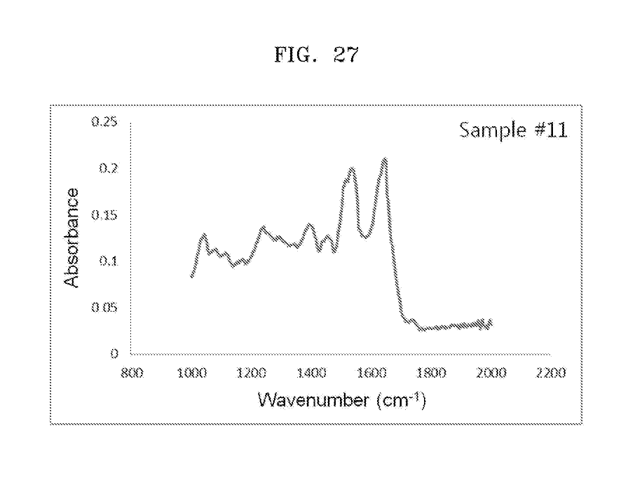

FIGS. 17 through 27 are graphs illustrating IR spectrum data that is obtained from tissue of the skin of a plurality of samples (people). The IR spectrum data may be MIR spectrum data. The IR spectrum data may be obtained by performing IR spectroscopic analysis on the tissue of the skin. The IR spectroscopic analysis may be MIR spectroscopic analysis.

FIG. 28 is a graph for illustrating a method of extracting information of creatine in the IR spectrum data of FIG. 17 (sample #1). The creatine includes a COOH functional group, a C═N functional group, and a C—N functional group. In the IR spectrum data, a wavenumber corresponding to the COOH functional group may range from 1690 cm⁻¹ to 1760 cm⁻¹, a wavenumber corresponding to the C═N functional group may range from 1650 cm⁻¹ to 1720 cm⁻¹, and a wavenumber corresponding to the C—N functional group may range from 1020 cm⁻¹ to 1250 cm⁻¹. Accordingly, the information of the creatine may be obtained by reading an intensity value, that is, an absorbance, corresponding to each wavenumber. For example, an absorbance value of the creatine may be obtained by reading an absorbance corresponding to 1740 cm⁻¹ and an absorbance corresponding to 1700 cm⁻¹ in the IR spectrum data. Also, the absorbance value of the creatine may be normalized by being divided by an absorbance corresponding to a reference wavenumber. A measurement difference between the samples may be offset through such normalization. The reference wavenumber may be, for example, 1450 cm⁻¹. The reference wavenumber for normalization may be changed in various ways. The same operation may be performed on all of the samples (see FIGS. 17 through 27).

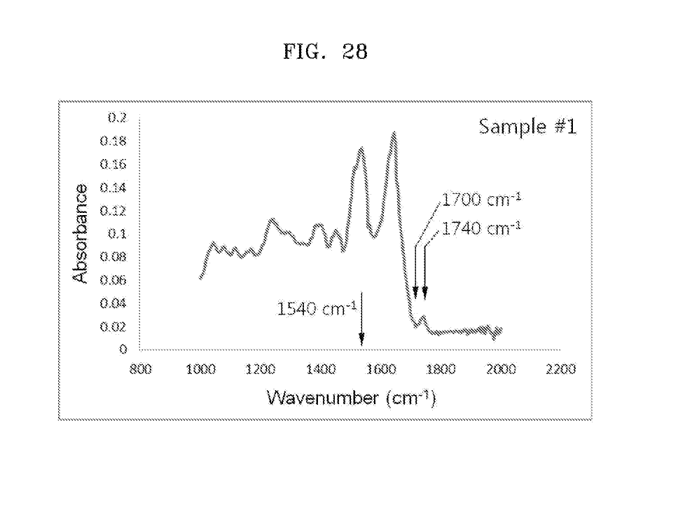

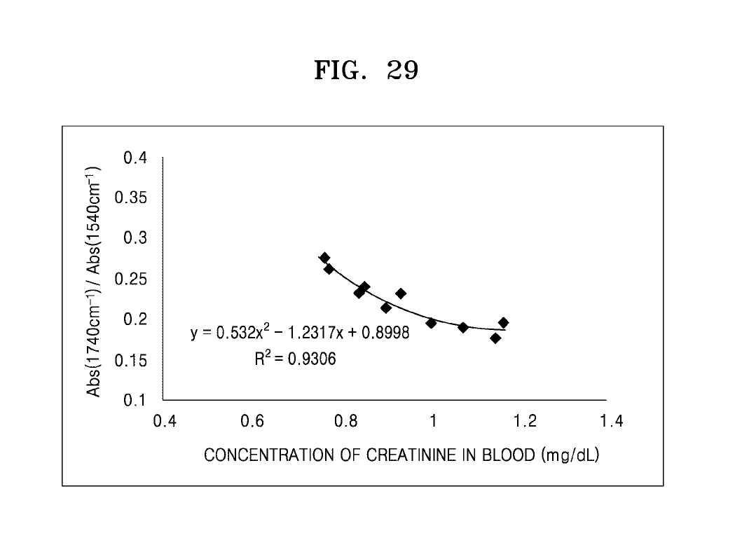

A concentration of creatinine in blood of each of the samples may be measured by collecting blood from each of the samples. When the concentration of the creatinine in the blood of each of the samples and the value of the creatine in the tissue of each of the samples obtained in data of FIGS. 17 through 27 are plotted, the graphs of FIGS. 29 and 30 may be obtained.

FIG. 29 is a graph illustrating a case where a Y-axis value is set to Abs (1740 cm⁻¹)/Abs (1540 cm⁻¹). That is, information of creatine is obtained by reading an intensity, that is, an absorbance, corresponding to 1740 cm⁻¹ in IR spectrum data and dividing the absorbance by an intensity, that is, an absorbance, corresponding to 1540 cm⁻¹. In this case, 1740 cm⁻¹ may be a wavenumber corresponding to a COOH functional group and 1540 cm⁻¹ may be a reference wavenumber for normalization. The Y-axis value Abs (1740 cm⁻¹)/Abs (1540 cm⁻¹) is obtained from the IR spectrum data of each of the samples (see FIGS. 17 through 27) and a concentration of creatinine in blood of each of the samples is measured, and then the two values are plotted.

FIG. 30 is a graph illustrating a case in which a Y-axis value is set to [Abs (1740 cm⁻¹)+Abs (1700 cm⁻¹)]/Abs (1540 cm⁻¹). That is, information of creatine is obtained by summing an absorbance corresponding to 1740 cm⁻¹ and an absorbance corresponding to 1700 cm⁻¹ in IR spectrum data and dividing a resultant value by an absorbance corresponding to 1540 cm⁻¹. In this case, 1740 cm⁻¹ may be a wavenumber corresponding to a COOH functional group, 1700 cm⁻¹ may be a wavenumber corresponding to a C═N functional group, and 1540 cm⁻¹ may be a reference wavenumber for normalization. The Y-axis value [Abs (1740 cm⁻¹)+Abs (1700 cm⁻¹)]/Abs (1540 cm⁻¹) is obtained from the IR spectrum data of each of the samples (see FIGS. 17 through 27) and a concentration of creatinine in blood of each of the samples is measured, and then the two values are plotted.

Referring to each of FIGS. 29 and 30, it is found that there is a clear correlation between a concentration (X-axis value) of creatinine in blood and a value (Y-axis value) of creatine in tissue obtained from a plurality of samples. The correlation may be expressed as a function. The correlation of FIG. 29 may be expressed as a functional equation ┌y=0.532x²−1.2317x+0.8998┘, and in this case, a square R² of a correlation coefficient is 0.9306. The correlation of FIG. 30 may be expressed as a functional equation ┌y=1.6048x²−3.4521x+2.3551┘, and in this case, a square R² of a correlation coefficient was 0.889.

A data processor of a noninvasive measuring apparatus according to an exemplary embodiment may have an algorithm based on the correlation. Accordingly, when a value, that is, a Y-axis value, of a first material is obtained from IR spectrum data, a value, that is, an X-axis value, of a second material may be calculated and/or derived from the correlation. For example, raw data, including information about the first material A (e.g., creatine), may be obtained by using the measurer MU13 of FIG. 8, and information about the second material B (e.g., creatinine) may be calculated/derived from the raw data by using a data processor of the processor PU10.

Although a method of obtaining a correlation between specific materials (e.g., creatine and creatinine) by using IR spectrum data has been exemplarily explained with reference to FIGS. 17 through 30, a type of a material and a type of data may be changed. For example, when an A material in tissue is changed to a B material and the B material is diffused through a blood vessel into blood, the A material in the tissue may be noninvasively detected and the B material in the blood may be quantized according to an embodiment. Specific examples of the A material and the B material may be respectively creatine and creatinine. Accordingly, even when the A material in the tissue is changed to the B material and the B material is diffused through the blood vessel into the blood, the spirit may apply to the A material and the B material. Also, although an intensity value corresponding to a wavenumber of a specific material (e.g., creatine) is read in order to extract information of the specific material in spectrum data in FIG. 28, the information about the specific material in tissue may be obtained by using various regression analysis methods using intensity information (absorbance information) about all spectrum wavelengths. That is, the specific material may be quantized by using regression analysis. Examples of the regression analysis may include a partial least square (PLS) method.

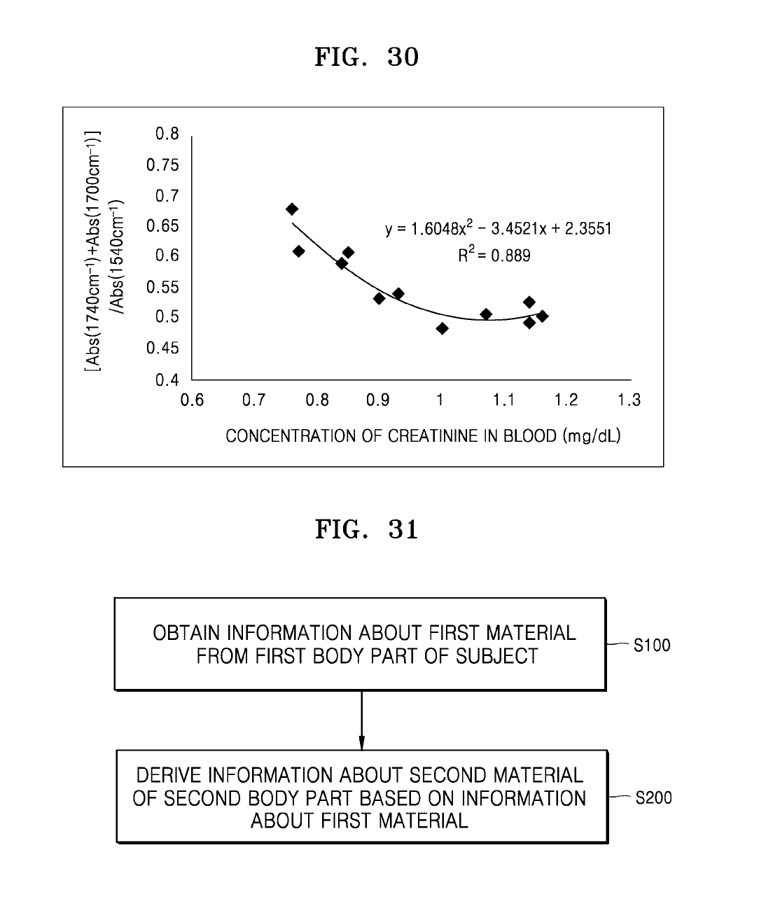

FIG. 31 is a flowchart for illustrating a method of noninvasively measuring a bio-analyte (hereinafter, referred to as a noninvasive measuring method), according to an exemplary embodiment. The description of the noninvasive measuring apparatuses 100A through 100F of FIGS. 1 through 30 applies to FIG. 31. Accordingly, the noninvasive measuring method of FIG. 31 may be understood based on the description of FIGS. 1 through 30.

Referring to FIG. 31, the noninvasive measuring method may include operation S100 in which information about a first material is obtained from a first body part of a subject and operation S200 in which information about a second material in a second body part of the subject is derived based on the information about the first material.

The first body part and the second body part may exist at different depths from the surface of the skin of the subject. For example, the first body part may exist at a first depth from the surface of the skin of the subject and the second body part may exist at a second depth, which is greater than the first depth, from the surface of the skin. For example, the first body part may be tissue and the second body part may be blood.

Operation S100 in which the information about the first material is obtained may include an operation in which the first body part is analyzed by using light. In this case, operation S100 in which the information about the first material is obtained may include an operation in which IR spectroscopic analysis is performed on the first body part. The IR spectroscopic analysis may be performed by using any one from among an IR spectrometer, an ATR-IR spectrometer, an FT-IR spectrometer, and an ATR-FTIR spectrometer. For example, the IR spectroscopic analysis may be performed by using any of the measurers MU10 through MU13 of FIGS. 3 through 9. The IR spectroscopic analysis may be performed by using MIR rays. That is, the IR spectroscopic analysis may be performed by using an MIR source. That MIR rays may have a wavelength ranging from about 2.5 μm to about 8 μm and may have a skin penetration depth ranging from about 50 μm to about 100 μm. The MIR rays may be effectively used to identify and quantize a component of a complex material by forming a narrow and sharp peak in spectrum data. However, the IR spectroscopic analysis may be performed by using a NIR source. Alternatively, operation S100 in which the information about the first material is obtained may include an operation in which Raman spectroscopic analysis is performed on the first body part. The Raman spectroscopic analysis may be performed by using, for example, the measurer MU20 of FIG. 10.

There may be a correlation between the first material and the second material, and operation S200 in which the information about the second material is derived may be performed by using an algorithm based on the correlation. Operation S200 in which the information about the second material is derived may be performed by using the processor PU10 of FIGS. 3 through 12. Before noninvasive measurement is performed, a correlation between the first material and the second material may be obtained and an algorithm based on the correlation may be established. As an alternative to the algorithm, the processor PU10 may use a correlation table of characteristic absorption of frequencies to identify functional groups (e.g., a COOH functional group, a C═N functional group, etc.) which are present in the first materials. The correlation table may include data on intensity of absorption peaks of the functional groups and wavenumbers of the absorption peaks. In addition, the correlation table may further include data on bandwidth of the absorption peaks. The processor PU10 may determine concentration of the second material based on the correlation table. A method of obtaining the correlation has already been described with reference to FIGS. 17 through 30.

The first material and the second material may be different materials that exist in different body parts (for example, the first body part and the second body part) of the subject. For example, the first material may include creatine or a constitute material of creatine, and the second material may include creatinine. Alternatively, the first material may include at least one from among a COOH functional group, a C═N functional group, and a C—N functional group, and the second material may include creatinine. When the first material includes at least one from among the COOH functional group, the C═N functional group, and the C—N functional group, in order to obtain the information about the first material, the noninvasive measuring method may include obtaining IR spectrum data about the first body part and reading an intensity value corresponding to at least one wavenumber range from among 1690 to 1760 cm⁻¹, 1650 to 1720 cm⁻¹, and 1020 to 1250 cm⁻¹ in the IR spectrum data. However, the first and second materials may be modified in various ways, and a method of obtaining the information about the first material may also be modified in various ways. For example, even when an A material (e.g., the first material) in tissue may be changed to a B material (e.g., the second material) and the B material is diffused into blood, the spirit may apply to the A material and the B material. Also, information about a specific material (e.g., the first material) in tissue may be obtained by using various regression analysis methods using intensity information (e.g., absorbance information) about all spectrum wavelengths.

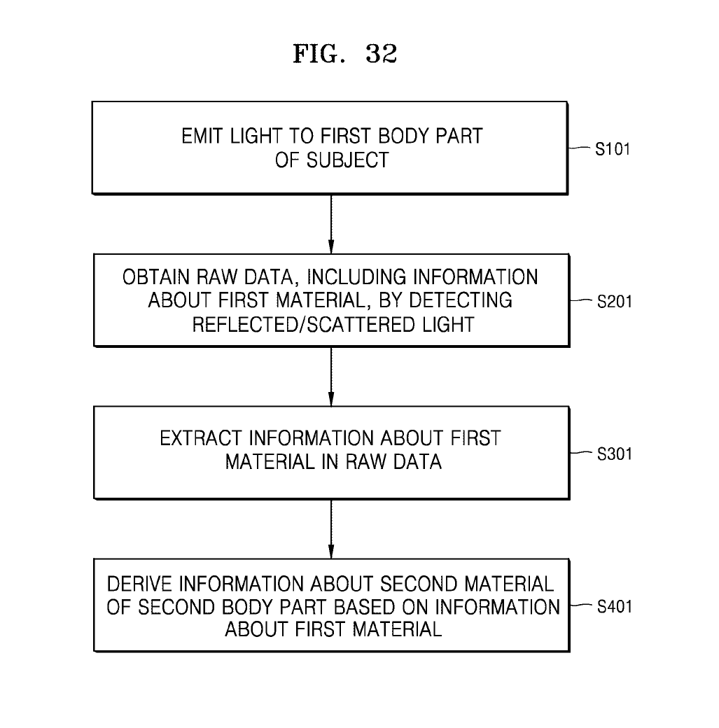

When the subject is measured by using light in a noninvasive measuring method according to an exemplary embodiment, the noninvasive measuring method may be performed as shown in FIG. 32. The noninvasive measuring method of FIG. 32 may be applied to, for example, the noninvasive measuring apparatuses 100A through 100F of FIGS. 3 through 5, 7, 8, and 10.

Referring to FIG. 32, the noninvasive measuring method may include operation S101 in which light is emitted to a first body part of a subject, operation S201 in which raw data, including information about a first material, is obtained by detecting light that is reflected or scattered by the first body part, operation S301 in which the information about the first material is extracted from the raw data, and operation S401 in which information about a second material in a second body part of the subject is calculated and/or derived from the extracted information about the first material. For example, when the noninvasive measuring apparatus 100A of FIG. 3 is used, the light source LS10 may perform operation S101, the detector D10 may perform operation S201, and the processor PU10 may perform operations S301 and S401.

Although not shown in FIG. 32, after operation S101 in which the light is emitted to the first body part of the subject, before the light that is reflected or scattered by the first body part is detected, the noninvasive measuring method may further include an operation in which the reflected or scattered light is dispersed. Also, after operation S401 in which the information about the second material is calculated and/or derived, the noninvasive measuring method may further include an operation in which the calculated and/or derived information about the second material is output.

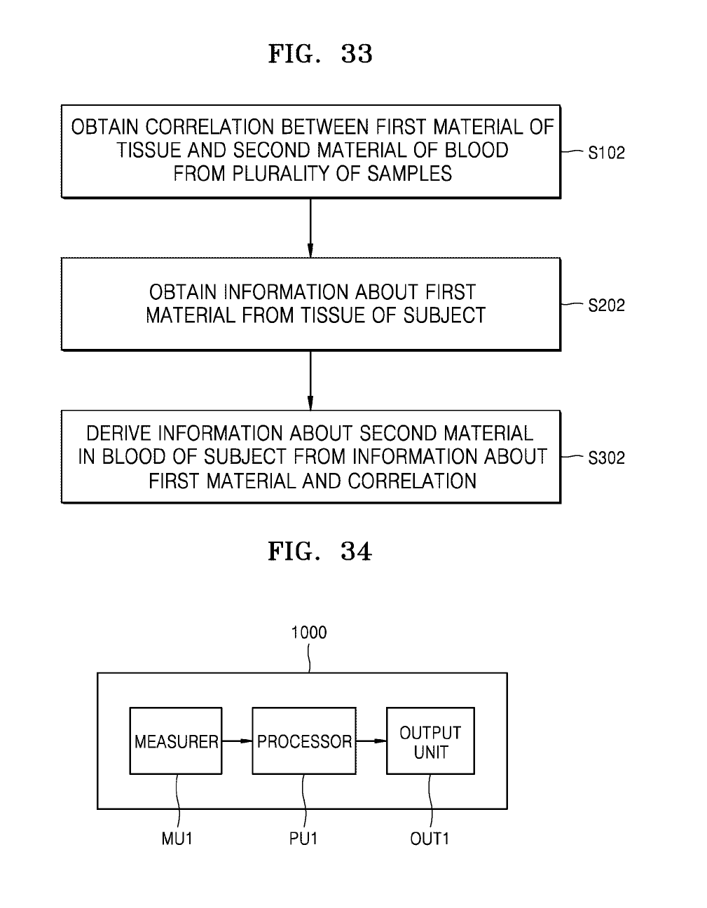

FIG. 33 is a flowchart for illustrating a noninvasive measuring method according to another exemplary embodiment. The description of the noninvasive measuring apparatuses 100A through 100F of FIGS. 1 through 30 applies to the method of FIG. 33. Accordingly, the noninvasive measuring method of FIG. 33 may be understood based on the description of FIGS. 1 through 30.

Referring to FIG. 33, the noninvasive measuring method may include operation S102 in which a correlation between a first material in tissue and a second material in blood is obtained from a plurality of samples, operation S202 in which information about the first material is obtained from the tissue of a subject, and operation S302 in which information about the second material in the blood of the subject is calculated and/or derived from the correlation between the information about the first material and the information about the second material.

More specifically, at operation S102, data about the first material in the tissue of each of the plurality of samples is obtained and data about the second material in the blood of each of the plurality of samples is obtained. In addition, a relationship between the data about the first material and the data about the second material is obtained. When the data about the first material is obtained from the plurality of samples, spectroscopy is used to obtain spectrum data from the tissue. Based on the spectrum data, an intensity value corresponding to the first material is determined. Also, when the data about the first material is obtained, normalization is performed by dividing the intensity value corresponding to the first material by an intensity value corresponding to a reference wavenumber. For example, operation S102 in which the correlation is obtained may be identical or similar to that described with reference to FIGS. 17 through 30.

At operation S202, IR spectroscopic analysis is performed on the tissue of the subject to obtain the information of the first material. The IR spectroscopic analysis may be performed by using any one from among an IR spectrometer, an ATR-IR spectrometer, an FT-IR spectrometer, and an ATR-FTIR spectrometer. The IR spectroscopic analysis may be performed by using MIR rays. Alternatively, the IR spectroscopic analysis may be performed by using NIR rays. Alternatively, operation S202 in which the information about the first material is obtained from the tissue of the subject may include an operation in which Raman spectroscopic analysis performed on the tissue of the subject. For example, operation S202 may include an operation in which the subject is measured/analyzed by using any of the measurers MU10 through MU13 and MU20 of FIGS. 3 through 10.

The first material and the second material may differ. For example, the first material may include creatine or a constituent material of creatine, and the second material may include creatinine. Alternatively, the first material may include at least one from among a COOH functional group, a C═N functional group, and a C—N functional group, and the second material may include creatinine. However, the first and second materials may be modified in various ways. For example, even when an A material (e.g., the first material) in tissue may be changed to a B material (e.g., the second material) and the B material may be diffused into blood, the spirit may apply to the A material and the B material.

Elements of a noninvasive measuring apparatus according to any of the embodiments, for example, a measurer, a processor, and an output unit, may be provided in one device or may be separately provided in at least two devices, which will be explained with reference to FIGS. 34 through 39.

FIG. 34 is a block diagram illustrating a noninvasive measuring apparatus according to an embodiment. Referring to FIG. 34, the noninvasive measuring apparatus may include a measurer MU1, a processor PU1, and an output unit OUT1 in one device 1000. The measurer MU1, the processor PU1, and the output unit OUT1 may be the same as those described with reference to FIGS. 3 through 12.

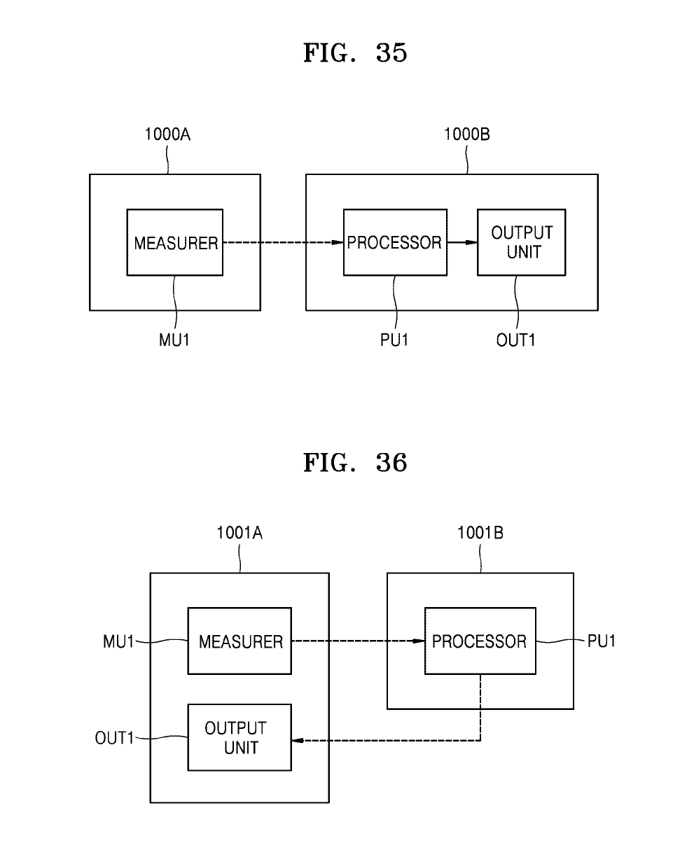

FIG. 35 is a block diagram illustrating a noninvasive measuring apparatus according to another exemplary embodiment. The noninvasive measuring apparatus may include the measurer MU1 in a first device 1000A, and may include the processor PU1 and the output unit OUT1 in a second device 1000B. A subject may be measured by the measurer MU1 of the first device 1000, and data obtained by the measurer MU1 may be transmitted to the processor PU1 of the second device 1000B. The measurer MU1 and the processor PU1 may be connected to each other through wireless communication or wired communication. To this end, a data receiver (not shown) for receiving the data may be further provided in the second device 1000B, and the data receiver may be connected to the processor PU1. Alternatively, the data receiver may be provided in the processor PU1. The data receiver may be referred to as a ‘data obtainer’.

FIG. 36 is a block diagram illustrating a noninvasive measuring apparatus according to another exemplary embodiment. Referring to FIG. 36, the noninvasive measuring apparatus may include the measurer MU1 and the output unit OUT1 in a first device 1001A, and may include the processor PU1 in a second device 1001B. A subject may be measured by the measurer MU1 of the first device A, and data obtained by the measurer MU1 may be transmitted to the processor PU1 of the second device 1001B. The measurer MU1 and the processor PU1 may be connected to each other through wireless communication or wired communication. Resultant data that is derived by the processor PU1 may be transmitted to the output unit OUT1 of the first device 1001A. The processor PU1 and the output unit OUT1 may also be connected to each other through wireless communication or wired communication.

FIG. 37 is a block diagram illustrating a noninvasive measuring apparatus according to another exemplary embodiment. Referring to FIG. 37, the noninvasive measuring apparatus may include the measurer MU1 and the processor PU1 in a first device 1002A, and may include the output unit OUT1 in a second device 1002B. A subject may be measured by the measurer MU1 of the first device 1002A, and data obtained by the measurer MU1 may be transmitted to the processor PU1. Resultant data that is derived by the processor PU1 may be transmitted to the output unit OUT1 of the second device 1002B. The processor PU1 and the output unit OUT1 may be connected to each other through wireless communication or wired communication.

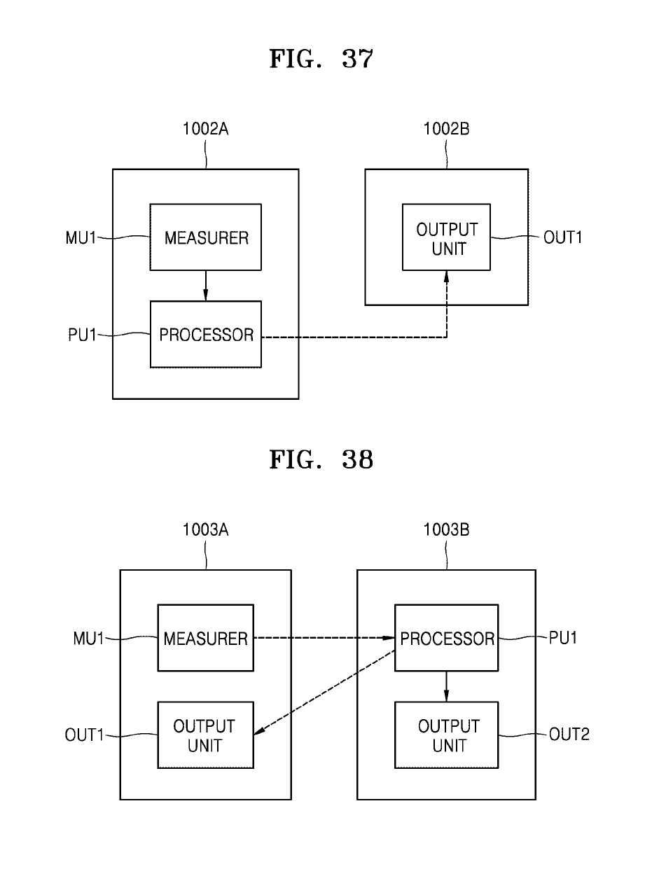

FIG. 38 is a block diagram illustrating a noninvasive measuring apparatus according to another embodiment. Referring to FIG. 38, the noninvasive measuring apparatus may include the measurer MU1 and the output unit (hereinafter, referred to as a first output unit in FIG. 38) OUT1 in a first device 1003A, and may include the processor PU1 and a second output unit OUT2 in a second device 1003B. A subject may be measured by the measurer MU1 of the first device 1003A, and data obtained by the measurer MU1 may be transmitted to the processor PU1 of the second device 1003B. Resultant data that is derived by the processor PU1 may be transmitted to the first output unit OUT1 of the first device 1003A and/or the second output unit OUT2 of the second device 1003B. The processor PU1 and the first output unit OUT1 may be connected to each other through wireless communication or wired communication.

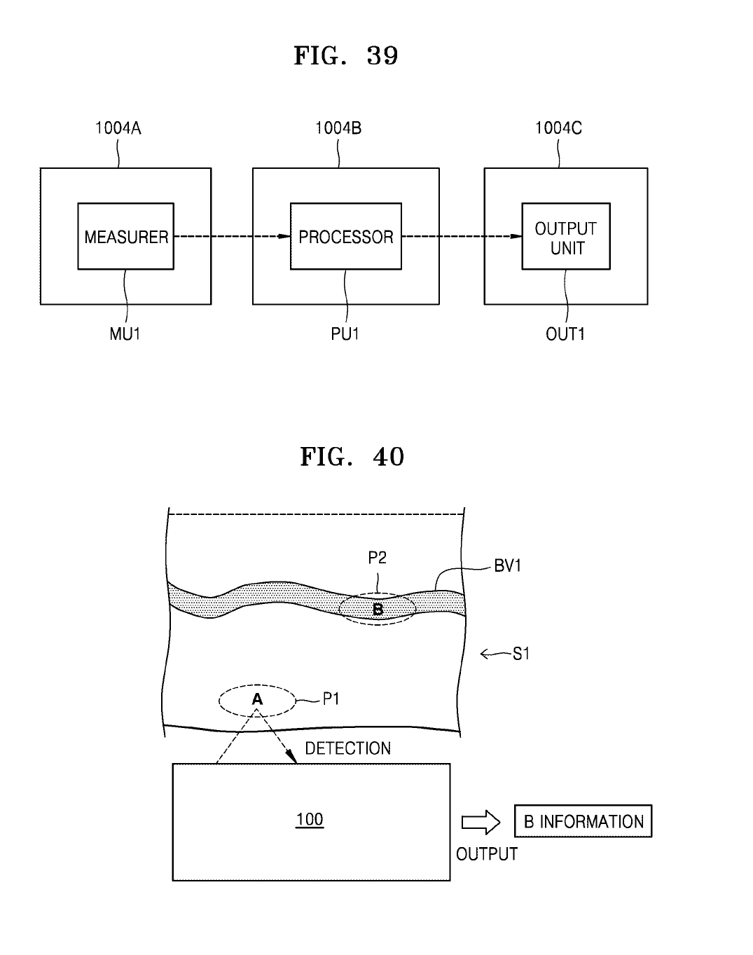

FIG. 39 is a block diagram illustrating a noninvasive measuring apparatus according to another embodiment. Referring to FIG. 39, the noninvasive measuring apparatus may include the measurer MU1 in a first device 1004A, may include the processor PU1 in a second device 1004B, and may include the output unit OUT1 in a third device 1004C. A subject may be measured by the measurer MU1 of the first device 1004A, and data obtained by the measurer MU1 may be transmitted to the processor PU1 of the second device 1004B. Resultant data that is derived by the processor PU1 may be transmitted to the output unit OUT1 of the third device. The measurer MU1 and the processor PU1 may be connected to each other through wireless communication or wired communication, and the processor PU1 and the output unit OUT1 may also be connected to each other through wireless communication or wired communication.

Any of the noninvasive measuring apparatuses of FIGS. 34 through 39 may be referred to as a ‘noninvasive measuring system’. The noninvasive measuring apparatus or the noninvasive measuring system may be applied to small-medium medical devices that are used in public places and small medical devices and health care devices that are possessed and carried by individuals as well as medical devices that are used in hospitals or health examination centers. Also, the noninvasive measuring apparatus or the noninvasive measuring system may be applied to mobile phones and peripheral devices (auxiliary devices) thereof.

In addition, although the noninvasive measuring apparatus 100 measures the subject S1 from above the subject S1 in FIG. 1, relative positions of the noninvasive measuring apparatus 100 and the subject S1 may be changed. For example, as shown in FIG. 40, the noninvasive measuring apparatus 100 may measure the subject S1 from below the subject S1. Relative positions of the noninvasive measuring apparatus 100 and the subject S1 may be modified in various other ways.

Also, as shown in FIG. 3, etc., when the subject S10 is detected by using light, a depth to which the light penetrates skin/tissue may be determined by a light source. When an A material in tissue (e.g., an epidermis or a dermis) is quantized in order to detect a B material in a blood vessel, a light source that generates light having a wavelength that penetrates only the tissue may be used or a filter that selectively filters (or receives) light that is scattered or reflected by the tissue may be used.

Also, although the subject S10 is mainly measured by using the light L10′ that is reflected or scattered by the first body part P10 of the subject S10 in FIG. 3, etc., according to other embodiments, the subject S10 may be measured by using light that passes through the subject S10. For example, a thin region, for example, an ear of a living creating/animal such as a human, may be measured by using light that passes through the thin region. Even in this case, IR spectroscopic analysis or Raman spectroscopic analysis may be used. The IR spectroscopic analysis may be performed by using MIR rays or NIR rays.

Also, although a first body part of a subject is mainly detected by using light (e.g., IR rays or a laser) in the above embodiments, a detection method may be modified. For example, the first body part of the subject may be detected by using an electrical signal, instead of light. For example, information about the first body part may be obtained by applying an electrical signal (e.g., a low voltage signal) to the first body part and then detecting a change in impedance. A method of detecting the first body part may be modified in various other ways.

In addition, the noninvasive measuring apparatus and the noninvasive measuring method may be applied to various analytes of various living creatures such as humans and animals, and may be used to measure and determine various diseases and health-related indices (information). For example, the noninvasive measuring apparatus and the noninvasive measuring method may be used to test diabetes, liver functions (alanine transaminase (ALT) levels, etc.), kidney functions, or metabolic syndromes. The types of a first material in a first body part and a second material in a second body part may vary according to diseases or functions. For example, in order to test liver functions (ALT levels, etc.), information of glutamate in tissue may be obtained and a concentration of gamma-glutamyl transpeptidase (γ-GTP) in blood may be derived from the information of the glutamate. Alternatively, information of hyaluronic acid in tissue may be obtained and a concentration of cortisol in blood may be derived from the information of the hyaluronic acid. Alternatively, information of cholesterol or cholesterol ester in tissue (e.g., an epidermis or a dermis) may be obtained and a concentration of low-density lipoprotein (LDL) cholesterol or high-density lipoprotein (HDL) cholesterol in blood may be derived from the information of the cholesterol or the cholesterol ester. In this case, the cholesterol or the cholesterol ester is different from the LDL cholesterol or the HDL cholesterol.

A subject may be very simply noninvasively examined by using the noninvasive measuring apparatus and the noninvasive measuring method. An invasive measuring method that is performed by collecting blood of the subject and measuring and analyzing the collected blood has disadvantages in that the subject feels pain when the blood is collected and reagents and colorimetric assays that react with a specific material of the blood have to be used when the blood is analyzed. However, according to any embodiment, for example, a target analyte in blood may be accurately (or relatively accurately) measured by just analyzing/detecting skin/tissue without collecting blood. Accordingly, various problems/disadvantages of the invasive measuring method may be solved.

In addition, there may be a first comparative method of noninvasively directly detecting an analyte in blood and a second comparative method of detecting an A material in tissue and indirectly measuring the A material in blood. However, the first comparative method has problems that feasibility is low because of complexity in a position and a structure of a blood vessel and the second comparative method has problems that the second comparative method may be used only when the A material in the blood is diffused into tissue and may not be used when a measurement signal of the A material in the tissue is weak. However, the noninvasive measuring apparatus and the noninvasive measuring method according to any of the embodiments may have higher feasibility, more detectable materials, and a higher SNR than the first and second comparative methods.

The foregoing exemplary embodiments are merely exemplary and are not to be construed as limiting. The present disclosure can be readily applied to other types of apparatuses. Also, the description of the exemplary embodiments is intended to be illustrative, and not to limit the scope of the claims, and many alternatives, modifications, and variations will be apparent to those skilled in the art. For example, it will be understood by one of ordinary skill in the art that a measurer that detects light that passes through a predetermined body part of a subject may be used or a measurer that analyzes a subject by detecting a change in impedance may be used. Also, any of the noninvasive measuring methods of FIGS. 17 through 33 may also be modified in various ways.

What is claimed is:

1. An apparatus for noninvasively measuring a bio-analyte, the apparatus comprising:
a sensor configured to obtain information of a first material from a first body part of a subject; and
a processor configured to obtain information about a second material in a second body part of the subject based on a correlation between the first material and the second material and the information of the first material.

2. The apparatus of claim 1, wherein the first body part and the second body part exist at different depths from a surface of a skin of the subject.

3. The apparatus of claim 1, wherein the first body part is tissue of the subject and the second body part is blood of the subject.

4. The apparatus of claim 1, wherein the first body part is an epidermis or a dermis of the subject.

5. The apparatus of claim 1, wherein the sensor is an optical data obtainer comprising:
a light source configured to emit light to the first body part of the subject; and
a detector configured to detect light that is reflected or scattered by the first body part.

6. The apparatus of claim 5, wherein the optical data obtainer further comprises a spectrometer configured to disperse the light that is reflected or scattered by the first body part and transmit the reflected or scattered light to the detector.

7. The apparatus of claim 1, wherein the sensor is an optical data obtainer comprising an infrared (IR) spectrometer configured to obtain the information of the first material from the first body part.

8. The apparatus of claim 7, wherein the IR spectrometer comprises at least one from among a mid-IR (MIR) spectrometer using MIR rays, an attenuated total reflectance (ATR)-IR spectrometer, a Fourier transform (FT)-IR spectrometer, and an ATR-Fourier transform infrared (FTIR) spectrometer.

9. The apparatus of claim 8, wherein the MIR rays have a wavelength ranging from about 2.5 μm to about 8 μm.

10. The apparatus of claim 1, wherein the sensor comprises a Raman spectrometer configured to obtain raw data comprising the information of the first material from the first body part.

11. The apparatus of claim 1, wherein the first material comprises creatine or a constituent material of creatine, and the second material comprises creatinine.

12. The apparatus of claim 1, wherein the first material comprises at least one from among a COOH functional group, a C═N functional group, and a C—N functional group, and the second material comprises creatinine.

13. The apparatus of claim 1, wherein the sensor is further configured to obtain IR spectrum data about the first body part to obtain the information of the first material and determine an intensity value corresponding to at least one wavenumber range from among 1690 to 1760 cm⁻¹, 1650 to 1720 cm⁻¹, and 1020 to 1250 cm⁻¹ in the IR spectrum data.

14. An apparatus for noninvasively measuring a bio-analyte comprising:
a sensor configured to noninvasively obtain information of a first material from a skin of a subject; and
a data processor configured derive information of a second material in blood of the subject from the information of the first material.

15. The apparatus of claim 14, wherein the sensor comprises at least one from among an infrared (IR) spectrometer, an attenuated total reflectance (ATR)-IR spectrometer, a Fourier transform (FT)-IR spectrometer, an ATR-Fourier transform infrared (FTIR) spectrometer, and a Raman spectrometer.

16. The apparatus of claim 15, wherein the measurer uses a mid-IR (MIR) light source.

17. A method of noninvasively measuring a bio-analyte comprising:
obtaining, by a sensor, information of a first material from a first body part of a subject; and
deriving information of a second material in a second body part of the subject based on a correlation between the first material and the second material and the information of the first material, by a processor.

18. The method of claim 17, wherein the first body part and the second body part exist at different depths from a surface of a skin of the subject.

19. The method of claim 17, wherein the first body part is tissue of the subject and the second body part is blood of the subject.

20. The method of claim 17, wherein the obtaining the information of the first material comprises analyzing the first body part by using light.

21. The method of claim 17, wherein the obtaining the information of the first material comprises performing infrared (IR) spectroscopic analysis on the first body part.

22. The method of claim 21, wherein the IR spectroscopic analysis is performed by using mid-IR (MIR) rays.

23. The method of claim 21, wherein the IR spectroscopic analysis is performed by using one from among an IR spectrometer, an attenuated total reflectance (ATR)-IR spectrometer, a Fourier transform (FT)-IR spectrometer, and an ATR-Fourier transform infrared (FTIR) spectrometer.

24. The method of claim 17, wherein the obtaining the information of the first material comprises performing Raman spectroscopic analysis on the first body part.

25. The method of claim 17, further comprising obtaining the correlation between the first material and the second material,
wherein the deriving the information of the second material is performed by using an algorithm based on the correlation.

26. The method of claim 17, wherein the first material comprises creatine or a constituent material of creatine, and the second material comprises creatinine.

27. A method of noninvasively measuring a bio-analyte comprising:
obtaining information of a first material from tissue of a subject; and
deriving information of a second material in blood of the subject from the information of the first material based on a correlation between the first material in tissue of a plurality of samples and the second material in blood of the plurality of samples.

28. The method of claim 27, further comprising obtaining the correlation and the obtaining the correlation comprises:
obtaining data that indicates an amount of the first material in the tissue of each of the plurality of samples;
obtaining data that indicates an amount of the second material in the blood of each of the plurality of samples; and
obtaining a relationship between the data of the first material and the data of the second material.

29. The method of claim 28, wherein the obtaining of the data that indicates the amount of the first material in the tissue of each of the plurality of samples comprises:
obtaining spectrum data by using spectroscopy in the tissue of each of the plurality of samples; and
determining an intensity value corresponding to the first material in the spectrum data.

30. The method of claim 29, wherein the obtaining the data of the first material in the tissue of each of the plurality of samples further comprises performing normalization by dividing the intensity value corresponding to the first material by an intensity value corresponding to a reference wavenumber.

31. An apparatus for measuring a bio-analyte of a subject, the apparatus comprising:
a spectrometer configured to collect light reflected from an area of interest on a surface of a skin of the subject; and
a processor configured to analyze the collected light to determine a concentration of a first component in the skin of the subject and determine a concentration of a second component beneath the skin surface of the subject based on a correlation between the concentration of the first component and the concentration of the second component.

32. The apparatus of claim 31, wherein the processor is further configured to determine the concentration of the second component based on a correlation table that comprises data on intensities of absorption peaks of a plurality of functional groups of the first material and wavenumbers of the absorption peaks.

33. The apparatus of claim 32, wherein the plurality of functional groups comprise at least one from among a COOH functional group, a C═N functional group, and a C—N functional group.
